(19) United States
(12) Patent Application Publication
(10) Pub. No.: US 2023/0102192 A1
(43) Pub. Date: Mar. 30, 2023

(54) THERAPIES FOR THE TREATMENT OF DISEASES AND DISORDERS ASSOCIATED WITH ABNORMAL EXPRESSION OF A NEURAL-ASSOCIATED GENE

(71) Applicant: The Regents of the University of California, Oakland, CA (US)

(72) Inventors: Alysson Renato Muotri, San Diego, CA (US); Cleber Augusto Trujillo, Cambridge, MA (US)

(21) Appl. No.: 17/799,597

(22) PCT Filed: Feb. 18, 2021
(86) PCT No.: PCT/US21/18573

§ 371 Date: Aug. 12, 2022

Related U.S. Application Data
(60) Provisional application No. 62/979,223, filed on Feb. 20, 2020.

(51) Int. Cl. A61K 31/4015; A61K 31/454; A61K 31/439; A61K 31/4995; A61K 31/4545; A61K 31/5377; A61K 31/501; A61K 31/5513; A61K 31/444; A61K 31/167; A61P 25/28; G01N 33/50

(52) U.S. Cl. CPC A61K 31/4015; A61K 31/454; A61K 31/439; A61K 31/4995; A61K 31/4545; A61K 31/5377; A61K 31/501; A61K 31/5513; A61K 31/444; A61K 31/167; A61P 25/28; G01N 33/5058; G01N 33/5023

(57) ABSTRACT

The disclosure provides methods for rescuing synaptic defects caused by abnormal MeCP2 expression in a subject in need thereof, comprising administering to the subject therapeutically effective amount(s) of a nootropic agent and/or an alpha-7 nicotinic acetylcholine receptor (α7-nAChR) agonist. The disclosure further provides methods for screening candidate drug candidates in a tiered series of assays and models (neurons, MECP2-mosaic neurospheres, and cortical organoids).

### CROSS REFERENCE TO RELATED APPLICATIONS

This application claims priority under 35 U.S.C. § 119 from Provisional Application Ser. No. 62/979,223, filed Feb. 20, 2020, the disclosures of which are incorporated herein by reference.

### TECHNICAL FIELD

The disclosure provides for the discovery and development of therapies that can rescue synaptic defects caused by abnormal expression of neural-associated gene, like MECP2.

### BACKGROUND

Duplication or deficiency of the X-linked MECP2 gene reliably produces profound neurodevelopmental impairment, and its mutation is almost universally responsible for Rett syndrome (RTT). Distinct MECP2 mutations and cellular mosaicism may underlie RTTs spectrum of symptomatic severity, and MECP2 mutations have also been detected in other neurological disorders with RTT phenotypic overlap. There remain, however, no clinically approved treatments for RTT, and therapeutic discovery for neurodevelopmental disorders in general has been hindered by limited ability to investigate disease pathophysiology in a human context. Human pluripotent stem cell technology offers a platform to identify neuropathology and test candidate therapeutics.

### SUMMARY

The disclosure provides for human stem cell-derived models that allow for the study of synaptic dysregulation downstream caused by a disruption of MECP2 expression. The disclosure further provides use of these models to screen for pharmacologic compounds that can abrogate or reverse this dysfunction in a targeted manner. More specifically, using a strategic series of increasingly complex in vitro models, two compounds were identified, Nefiracetam and PHA 543613, that specifically rescued the cytologic neuropathology caused by MECP2 knockout. The capacity of these compounds to reverse neuropathologic phenotypes in human models supports clinical studies for neurodevelopmental disorders in which MeCP2 deficiency is the predominant etiology.

The disclosure provides a method for treating or rescuing synaptic defects caused by abnormal MeCP2 expression in a subject in need thereof, comprising administering to the subject therapeutically effective amount(s) of a nootropic agent and/or an alpha-7 nicotinic acetylcholine receptor (α7-nAChR) agonist. In one embodiment, the nootropic is a racetam-based nootropic agent. In a further embodiment, the racetam-based nootropic agent is selected from nefiracetam, piracetam, aniracetam, oxiracetam, pramiracetam and phenylpiracetam. In still a further embodiment, the racetam-based nootropic agent is nefiracetam. In yet another or further embodiment of any of the foregoing, the α7-nAChR agonist is selected from N-[(3R)-1-azabicyclo[2.2.2]oct-3-yl]furo[2,3-c]pyridine-5-carboxamide (PHA-543613), bradanicline, encenicline, N-[(3R,5R)-1-azabicyclo[3.2.1]oct-3-yl]furo[2,3-c]pyridine-5-carboxamide (PHA-709829), (4-bromophenyl) 1,4-diazabicyclo[3.2.2]nonane-4-carboxylate (SSR-180), tropisetron, N-[(3S)-1-azabicyclo[2.2.2]oct-3-yl]-1H-indazole-3-carboxamide hydrochloride (RG3487), 1-[6-(4-fluorophenyl)pyridin-3-yl]-3-(4-piperidin-1-ylbutyl) urea (SEN34625/WYE-103914), N-[4-(3-pyridinyl)phenyl]-4-morpholinepentanamide (SEN12333), 5-(6-[(3R)-1-azabicyclo[2,2,2]oct-3-yloxy]pyridazin-3-yl)-1H-indole (ABT-107), clozapine, 3-(2,4-dimethoxy-benzylidene)anabaseine (GTS-21), semapimod, and (2S)-2′H-spiro[4-azabicyclo[2.2.2]octane-2,5′-[1,3]oxazolidin]-2′-one (AR-R17779). In still another or further embodiment of any of the foregoing, the α7-nAChR agonist is PHA-543613. In another or further embodiment of any of the foregoing, the nootropic agent and the alpha-7 nicotinic acetylcholine receptor (α7-nAChR) agonist are sequentially administered. In a further embodiment, a first pharmaceutical formulation comprises the nootropic agent and a second pharmaceutical formulation comprises the alpha-7 nicotinic acetylcholine receptor (α7-nAChR) agonist. In another or further embodiment, the nootropic agent and the alpha-7 nicotinic acetylcholine receptor (α7-nAChR) agonist are concurrently administered. In a further embodiment, a single pharmaceutical composition formulated for oral delivery comprises the nootropic agent and the alpha-7 nicotinic acetylcholine receptor (α7-nAChR) agonist. In yet another or further embodiment of any of the foregoing embodiments, the abnormal MeCP2 expression is cause by a genetic disease or disorder that affects the expression of the MECP2 gene. In a further embodiment, the genetic disease or disorder is selected from Rett syndrome, MECP2 duplication syndrome, neonatal encephalopathy, systemic lupus erythematosus (SLE), and autism spectrum disorder (ASD). In still a further embodiment, the genetic disease or disorder is Rett syndrome. In yet another embodiment of any of the foregoing embodiments, the subject is a female subject. In yet another embodiment of any of the foregoing embodiments, the subject is a male subject. In a further embodiment, the subject is less than 25 years of age. In still a further embodiment, the subject is less than 10 years of age.

The disclosure also provides a human-based neural drug screening platform for identifying therapeutic compounds that can ameliorate or rescue deleterious biological effect(s) resulting from abnormal expression of a gene from an X-chromosome, comprising (a) contacting set(s) of neurons that have been differentiated from human stem cells with a candidate drug, wherein a first set of neurons have been differentiated from human pluripotent stem cells that have mutation(s) affecting the normal expression of a gene from an X-chromosome and optionally, a second set of neurons that are differentiated from pluripotent stem cells that do not have said mutation(s); (b) contacting set(s) of neurospheres that have been derived from human stem cells with the candidate drug, wherein a first set of neurospheres comprises neurospheres that have been differentiated from human pluripotent stem cells that have mutation(s) affecting the normal expression of the gene from an X-chromosome, and optionally, a second set of neurospheres that have been differentiated from the human pluripotent stem cells that do not have said mutation(s); and/or (c) contacting set(s) of cortical organoids that have been generated from human stem cells with the candidate drug, wherein a first set of cortical organoids have been differentiated from human pluripotent stem cells that have mutation(s) affecting the normal expression of the gene from an X-chromosome, and optionally, a second set of cortical organoids that have been differentiated from the human pluripotent stem cells that do not have said mutation(s); (d) evaluating whether the candidate drug rescues or ameliorates rescue deleterious biological effect(s) resulting from abnormal expression of a gene from an X-chromosome in the first set of neurons, and/or the first set of neurospheres, and/or the first set of cortical organoids. In another embodiment, the method further comprises determining whether the candidate drug exhibits toxicity or negatively effects neural activity in the second set of neurons, and/or second set of neurospheres, and/or second set of cortical organoids. In another or further embodiment, the mutation(s) affecting the normal expression of a gene from an X-chromosome are loss-of-function mutations. In yet another embodiment of any of the foregoing embodiments, the gene is selected from MECP2, NLGN3, NLGN4, lysosomal alpha-galactosidase A, KDM6A, RPS6KA3, ABCD1, ATP7A, L1CAM, ATRX, and PHF8. In a further embodiment, the gene is MECP2. In still a further embodiment the biological activity of the neurons, neurospheres, and/or cortical organoids being determined is calcium oscillations, synaptic activity, and/or synaptic morphology. In yet a further embodiment, the first set of neurospheres is mosaic by further comprising neurospheres that have been differentiated from the human pluripotent stem cells that do not have said mutation(s).

### DESCRIPTION OF DRAWINGS

FIG. 1A-I demonstrates that human MECP2-KO neurons exhibit alterations in synapse-relevant genes and pathways. (A) Pluripotent stem cells (PSCs) with one of two distinct exonic loss-of-function mutations (Q83X nonsense or K82 frameshift (K82fs)) at the MECP2 locus were generated and differentiated to neurons via a neural progenitor cell (NPC) intermediate. (B) Immunofluorescent staining confirmed the absence of MeCP2 in MECP2-KO PSCs, NPCs, and neurons. (C, D) PCR array revealed differential genetic expression between control and MECP2-KO 28-day neurons, with many of the involved genes relevant to synaptic function (C). Expression of select genes on days 14 and 28 post differentiation (D). (E-G) Single-cell qPCR analysis distinguished populations of control and KO neurons (E) and showed altered expression of genes relevant to synaptic function (F) that overlapped with the results obtained via PCR array (G). (H) Left: Schematic of laminar cortical markers. Right: Quantitation of laminar cell markers (two-way analysis of variance (ANOVA) showed interaction F_{3, 24}=3.31, P=0.04, between cortical layers, F_{3, 24}=16.84, P<0.0001, and genotype, F_{1, 24}=14.40, P=0.0009, with difference in the proportion of layers V and VI, 95% CI [8.31, 42.41]) and altered proportions of neurotransmitter markers (two-way ANOVA showed interaction F5, 24=27.33, P<0.0001, between neurotransmitter, F5, 24=1610, P<0.0001, and genotype, F1, 24=86.46, P<0.0001, with different proportions of glutamatergic, 95% CI [22.33, 33.60], and cholinergic, 95% CI [0.166, 11.43], cells). (I) Gene ontology (GO) analysis affirmed that differences between MECP2-KO and control neurons concentrated in pathways relevant to synaptic function.

FIG. 2A-I shows the generation of MECP2-KO pluripotent stem cells. (A) Mutations resulting in loss of MECP2 (SEQ ID Nos: 1, 2 and 3). (B) CRISPR guide RNA to generate K82frameshift mutation. (C) Identification of off-target mutations. (D-I) MECP2-KO was successful and did not affect pluripotency, as confirmed by immunochemistry (D), teratoma assay (E), Western blot (F), karyotype (G), and RT-qPCR (H, I).

FIG. 3A-E demonstrates that MECP2-KO neurons exhibit defects in synaptogenic morphology. (A) Schematic showing neuronal differentiation from human PSCs. (B, C) No difference in the resultant proportions of neurons or glia was observed between MECP2-KO and control neuronal differentiations (B). Proportions of glutamatergic and gabaergic neurons (C). (D) MECP2-KO neurons had decreased soma area and spine number (t24=5.03, P<0.0001). (E) Observation over time showed decreased spine stability (P<0.01), but spine motility (P=0.17), spine formation (P=0.26), and spine length (P=0.28) were unchanged. Analyses with two-sided Student's t-tests. Data are presented as mean±s.e.m.

FIG. 4A-K provides a PCR array and single-cell qPCR analysis. (A, B) RT-PCR array shows differences in genetic expression become increasingly pronounced with neuronal differentiation. (C-K) Single-cell RT-qPCR analysis likewise shows differences in genetic expression due to MECP2-KO that appear to involve synaptic function and become more pronounced upon neuronal differentiation of neural progenitor cells.

FIG. 5A-I presents screening assays of select drugs with synaptic action in human neurons which identified Nefiracetam and PHA 543613 as top treatment candidates for MeCP2 deficiency. (A) In situ neural network modeling with previous synaptic puncta values for untreated (top, KD [6/16]=0.375*control) and treated (bottom, KD [24/16]=1.5*control) RTT neurons (18) suggests that isolated increase of synaptic knockdown sufficiently increases activity (untreated, mean=0.665, range=[0.050, 1.002]; treated, mean=1.542, range=[0.997, 8.395]). (B, C) List of pharmaceuticals for screening (B). Schematic of drug treatment workflow: one-month old neurons were treated for two weeks and then assayed (C). (D, E) Western blot analyses show presynaptic Synapsin1 (D) and postsynaptic PSD-95 (E) protein content is decreased in untreated MECP2-KO neurons compared to controls and can be significantly increased by drug treatment (F15, 32=3.883, P<0.0006 and F15, 32=3.883, P<0.0006 for Synapsin1 and PSD-95, respectively). For full Western blot see fig. S4A. (F, G) MECP2-KO neurons show a pharmaceutically rescuable reduction of co-localized synaptic puncta compared to control neurons (one-way ANOVA, F16, 118=9.148, P<0.0001; Dunnet's multiple comparison test versus KO: Carbamoylcholine, P<0.05; Acamprosate, P<0.01; Nefiracetam and control, P<0.001). (H) Drug treatment increases calcium oscillation frequency (one-way ANOVA, F4, 52=20.28, P<0.0001, Dunnett's multiple comparison test vs KO: Nefiracetam, P<0.01; PHA 543613, P<0.001; Acamprosate, P<0.001) and the percentage of active neurons (one-way ANOVA, F4, 52=23.11, P<0.0001; Dunnett's multiple comparison test vs KO: Nefiracetam, P<0.001; PHA 543613, P<0.01; Acamprosate, P<0.01). (I) Treatment with either Nefiracetam or PHA 543613 increases network spiking activity in MECP2-KO neurons on multi-electrode array (one-way ANOVA, F4, 20=2.531, P=0.073). Error bars represent standard deviation.

FIG. 6A-G shows the treatment of MECP2-KO neurons. (A, B) Representative example of full Western blot for drug treatment (A) and analysis of treatment effect on quantities of the astrocytic marker GFAP and neuronal marker MAP2 (B). (C) Full depiction of synaptic puncta following drug treatment. (D) Synaptic puncta co-localization following drug treatment of control neurons (one-way ANOVA, F6, 49=10.94, P<0.0001). (E, F) Calcium imaging showed increasing spike frequency with time (E) that was tractable to manipulators of synaptic function (F). (G) Representative image of MEA activity of control and MECP2-KO neurons. Data are presented as mean±s.e.m.

FIG. 7A-K provides for the treatment of MECP2-KO mosaic neurospheres with Nefiracetam and PHA 543613. (A) Schematic showing the formation of control, MECP2-KO, and mosaic MECP2-KO/control neurospheres (WT83/Q83X cell lines were used). (B, C) Immunofluorescent staining (B) and quantitation (C) confirmed the graded decrease in MeCP2 expression from control to 50% MECP2 mosaic to full MECP2-KO neurospheres. Scale bar=50 μm. (D) 8-week MECP2-KO neurospheres (N=44) exhibited decreased size (diameter; *P<0.01), but 50% MECP2-mosaic neurospheres (CTR/KO; N=44) were unchanged from controls (one-way ANOVA, F_{2, 150}=6.03, P=0.003; Dunnett's multiple comparisons test; N=65). Left: representative brightfield images of neurospheres; Right: neurosphere size quantification. Scale bar=200 μm. (E) Calcium imaging analysis showing calcium transients normalized by control (one-way ANOVA, F_{2, 72}=15.62, P<0.0001; Dunnett's multiple comparisons test vs control: MECP2-mosaic, P<0.001; MECP2-KO, P<0.001). WT83/Q83X cell lines were used; N=44 Q83X neurospheres and 65 WT83 neurospheres. (F) 8-week neurosphere size (diameter) remained unchanged by treatment with either drug (one-way ANOVA, F_{6, 344}=0.52, P=0.79; N=44 Q83X neurospheres and 65 WT83 neurospheres). (G) Cell viability was improved or unharmed in treated 8-week neurospheres (one-way ANOVA, F_{9, 104}=6.81; Dunnett's multiple comparisons test vs CTR/KO untreated: Nefi+PHA 1 μM, P<0.01 and Z=2.974; Nefi+PHA 10 μM, P<0.001 and Z=3.733; N=8-24 neurospheres per condition). (H, I) Calcium transient frequency (number of peaks in 10 mins recording) normalized by CTR/KO untreated (H; one-way ANOVA, F_{9, 239}=6.18; Dunnett's multiple comparisons test vs CTR/KO untreated: Nefi 1 μM, P<0.05 and Z=0.780; Nefi+PHA 10 μM, P<0.001 and Z=1.972; N=15-61 neurospheres per condition) and calcium transient amplitude normalized by CTR/KO untreated (I; one-way ANOVA, F_{9, 241}=1.27, P=0.255; N=16-62 neurospheres per condition). (J, K) MEA spike frequency heatmap (J) and quantification (K) normalized by CTR/KO untreated (one-way ANOVA, F_{5, 39}=1.21, P=0.321; Dunnett's multiple comparisons test vs CTR/KO untreated: PHA, P=0.65 and Z=1.134; Nefi, P=0.98 and Z=0.06; PHA+Nefi, P=0.86 and Z=0.94; N=7-8 wells per condition). ‘X’ on activity heatmap signifies absence of a neurosphere in that position. Data information: Nefi: Nefiracetam; PHA: PHA 543613. Note that * in F-K signifies a statistically significant difference in treated MECP2-mosaic (CTR/KO) neurospheres compared to untreated CTR/KO neurospheres. Data are presented as mean±s.e.m.

FIG. 8A-D provides a comparison of MECP2-KO, 50% MECP2-KO mosaic, and control neurospheres. (A, B) MECP2-KO neurospheres exhibited decreased size (P<0.01), but 50% MECP2-mosaic neurospheres were unchanged from controls (one-way ANOVA, F2, 150=6.03, P=0.003; Dunnett's multiple comparison test). (C, D) Calcium imaging showing calcium peak frequency (C; one-way ANOVA, F2, 73=33.38, P<0.0001; Dunnett's multiple comparison test vs control: MECP2-mosaic, P<0.001; MECP2-KO, P<0.001) and calcium peak amplitude (D; one-way ANOVA, F2, 72=15.62, P<0.0001; Dunnett's multiple comparison test vs control: MECP2-mosaic, P<0.001; MECP2-KO, P<0.001). Data are presented as mean±s.e.m.

FIG. 9A-M shows that treatment increases synaptogenic expression and network activity in MECP2-KO cortical organoids. (A) Schematic of PSC aggregation and development into cortical organoids. (B) 2-month old cortical organoid diameter (one-way ANOVA, F_{2, 173}=91.07; Dunnett's multiple comparisons test vs untreated: Nefiracetam, *P<0.001 and Z=5.414; PHA 543613, *P<0.001 and Z=5.351; WT83/Q83X and WT82/K82fs cell lines were used; N=28-90 organoids per condition). Left: representative brightfield images of cortical organoids; Right: cortical organoid size quantification. Scale bar=200 μm. (C) Ki67+ proliferative area (Student's t-test, t₁₀=0.36, P=0.73, N=6 each). Left: representative immunofluorescence images of progenitor, proliferative, and neuronal markers in cortical organoids; Right: quantification of Ki67+ proliferation in cortical organoids. Scale bar=50 μm. (D, E) RNA sequencing (D, left: heatmap) and GO analysis (D, right) of untreated MECP2-KO organoids vs Nefiracetam (N=4 samples each of ˜10-15 pooled organoids) indicated that treatment upregulated genetic expression in synapse-relevant pathways. E, center band of the box is median log 2 normalized expression and edges are 25^{th} and 75^{th} percentiles; whiskers are minimum and maximum log 2 normalized expression values. (F, G) RNA sequencing (F, left: heatmap) and GO analysis (F, right) of untreated MECP2-KO organoids vs PHA 543613 (N=4 samples each of ˜10-15 pooled organoids) indicated that treatment upregulated expression of genes in synapse-relevant pathways. G, center band of the box is median log 2 normalized expression and edges are 25^{th} and 75^{th} percentiles; whiskers are minimum and maximum log 2 normalized expression values. (H) Synapsin1+ puncta quantitation (one-way ANOVA, F_{3, 34}=10.07; Dunnett's multiple comparisons test vs untreated: Nefiracetam, *P<0.01 and Z=2.998; PHA 543613, P>0.05 and Z=1.266; WT83/Q83X cell lines were used; N=8-10 each). Left: representative image of Synapsin1+ immunofluorescent staining; Right: Synapsin1+ puncta quantification. Scale bar=20 μm. (I, J) MECP2-KO and control organoids had similar proportions of cells positive for the neuronal marker NeuN (I; Student's t-test, t₁₄=0.26, P=0.80, N=8 each) and cortical layer V and VI neuronal marker CTIP2 (J; one-way ANOVA, F_{3, 40}=2.489, P=0.074 and Z<1, WT83/Q83X and WT82/K82fs cell lines were used; N=8-14 organoids each). J, left: representative image of CTIP2+ immunofluorescent staining in cortical organoids; J, right: quantification of CTIP2+ neurons. Scale bar=50 μm. (K-M) MECP2-KO cortical organoids plated on MEA (K; left: schematic depicts process of plating organoids on MEA; right: top-down view of organoid on MEA; scale bar=200 μm) showed decreased population spiking (L; left and center: representative population spiking traces of control and MECP2-KO organoids; right: quantification of organoid population spiking activity, N=6 replicates each). Drug treatment increased spiking network activity of MECP2-KO cortical organoids to a level not significantly different from that of control organoids (M; one-way ANOVA, F_{3, 31}=3.775, Dunnett's multiple comparisons test vs control: untreated KO, *P<0.01 and Z=3.192; Nefiracetam, P=0.98 and Z=2.117; PHA 543613, P=0.12 and Z=1.101, WT83/Q83X cell lines were used; N=7-10 MEA wells per condition). Data information: Note that * signifies a statistically significant difference from untreated MECP2-KO cortical organoids. Data are presented as mean±s.e.m.

FIG. 10A-G demonstrates the generation and characterization of cortical organoids. (A) Schematic of the protocol used to generate cortical organoids. Scale bar=200 μm. (B) Reproducibility of organoid size at 3 weeks of maturation (N=20 independent experiment, 7 different cell lines). (C, D) Gene expression of specific neurotransmission markers of treated vs untreated cortical organoids (N=8 samples of treated organoids and 8 samples of control organoids, ˜10-15 organoids pooled per sample). Boxplot: center band of the box is median log 2 normalized expression and edges are 25^{th} and 75^{th} percentiles; whiskers are minimum and maximum log 2 normalized expression values. (E, F) Representative images of quantification of Synapsin1 puncta. Scale bar=50 μm. SYN1 puncta mask was made using ImageJ to access the number and area of the puncta. (G) Nefiracetam and PHA 543613 treatment did not rescue MECP2-KO neuronal morphology, for either the number of neurites (Kruskall-Wallis test, P=0.011; Dunn's multiple comparisons test vs KO untreated: control, P=0.01 and Z=3.13; Nefiracetam, P=0.49 and Z=1.74; PHA 543613, P>0.99 and Z=0.87; WT83/Q83X and WT82/K82fs cell lines; N=5-10 neurons per condition) or the total neurite length (one-way ANOVA, F_{3, 21}=9.62, P=0.0003; Dunnett's multiple comparisons test vs KO untreated: control, P=0.002 and Z=3.08; Nefiracetam, P=0.89 and Z=0.64; PHA 543613, P=0.99 and Z=0.17; WT83/Q83X and WT82/K82fs cell lines; N=5-10 neurons per condition) and, nearly uniformly, did not rescue nuclei size in neurons (one-way ANOVA, F_{3, 221}=26.04, P<0.0001; Dunnett's multiple comparisons test vs KO untreated: control, P<0.0001 and Z=6.37; Nefiracetam, P=0.91 and Z=0.39; PHA 543613, P=0.19 and Z=1.74; WT83/Q83X and WT82/K82fs cell lines; N=45 nuclei per condition), neurospheres (one-way ANOVA, F_{5, 264}=5.579, P<0.0001; Dunnett's multiple comparisons test vs CTR/KO untreated: control, P<0.0001 and Z=3.782; KO untreated, P=0.51 and Z=1.066; Nefiracetam, P=0.23 and Z=1.35; PHA 543613, P=0.99 and Z=0.05; Nefi+PHA, P=0.24 and Z=1.012; WT83/Q83X cell lines; N=45 nuclei per condition), or cortical organoids (one-way ANOVA, F_{3, 281}=6.594, P=0.0003; Dunnett's multiple comparisons test vs KO untreated: control, P=0.009 and Z=2.38; Nefiracetam, P=0.01 and Z=3.05; PHA 543613, P=0.98 and Z=0.30; WT83/Q83X and WT82/K82fs cell lines; N=45-90 nuclei per condition). Spine density was rescued or partially rescued by the compounds (one-way ANOVA, F_{3, 63}=8.449, P<0.0001; Dunnett's multiple comparisons test vs KO untreated: control, P<0.0001 and Z=4.35; Nefiracetam, P=0.16 and Z=2.01; PHA 543613, P=0.001 and Z=3.24; WT83/Q83X and WT82/K82fs cell lines; N=11-28 neurons per condition). Note that statistical comparisons are presented relative to KO untreated or, for mosaic neurospheres, CTR/KO untreated. Data information: Data are presented as mean±s.e.m.

### DETAILED DESCRIPTION

As used herein and in the appended claims, the singular forms “a,” “an,” and “the” include plural referents unless the context clearly dictates otherwise. Thus, for example, reference to “a prodrug” includes a plurality of such prodrugs and reference to “the chemotherapeutic agent” includes reference to one or more chemotherapeutic agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this invention is not limited to the particular methodology, protocols, and reagents, etc., described herein and as such may vary. The terminology used herein is for the purpose of describing particular embodiments only and is not intended to limit the scope of the invention, which is defined solely by the claims.

Other than in the operating examples, or where otherwise indicated, all numbers expressing quantities of ingredients or reaction conditions used herein should be understood as modified in all instances by the term “about.” The term “about” when used to described the invention, in connection with percentages means±10%, 5%, or typically 1%.

MECP2 is an X-linked gene that encodes an epigenetic regulatory protein, methyl CpG binding protein-2, that is critical for typical human brain development. In addition to an array of other neurodevelopmental disorders, MECP2 loss-of-function mutations are the most common etiology of RTT, a severe neurodevelopmental disorder clinically characterized by head growth deceleration, profound cognitive decline, regression of acquired abilities, and stereotypies in early infancy. Critically, these syndromic clinical features appear following a brief period of normal development. Although the precise neurobiological changes linking MECP2 mutation to the RTT phenotype are unclear, human postmortem tissue from RTT females portrays reduced brain size, decreased dendritic arborization and spine formation, and reduced synapse numbers. Moreover, studies in murine models of Mecp2-deficiency implicate an accordant deficit in synaptic function, including decreased synaptic transmission and plasticity. The findings to date thus suggest that synaptic dysfunction is a central pathology of MeCP2 deficiency and may be a viable treatment target.

Therapeutically targeting pathways downstream from MECP2 has been proposed as an RTT treatment strategy, and the narrow window of typical development suggests intervention during this period to bolster synaptic function may be opportune to preserve clinical function and ameliorate subsequent decline. Development and discovery of therapeutics for RTT and other neurodevelopmental disorders, however, has been challenged by limited opportunity to investigate disease pathogenesis in a human model. Advances in pluripotent stem cell (PSC) technology, including three-dimensional neural differentiation, offer a promising human-based platform to evaluate candidate therapeutics for neurodevelopmental disorders and hasten clinical translatability.

A series of increasingly complex human stem cell-derived models as a screening platform were used herein to identify pharmacologic compounds capable of specifically improving the neurocytological deficits caused by knockout of MECP2 (MECP2-KO) without affecting controls. The studies presented herein demonstrate MECP2-KO-attributable synaptic pathology in genetic expression, morphology, and physiology. More specifically, the analyses of MECP2-KO neurons revealed altered expression of synapse-relevant genes, indicating that synaptic impairment is the preeminent pathobiology of RTT. After identifying targetable neuropathology, an innovative, human-based drug screening platform was developed and utilized. This human-based drug screening platform was comprised of increasingly complex models that approach human fetal neurodevelopment in their recapitulative accuracy, thereby facilitating clinical translatability. A tiered series of assays and models (neurons, MECP2-mosaic neurospheres, and cortical organoids) were strategically employed to screen 14 targeted candidate pharmacologic compounds. Two of these compounds, Nefiracetam and PHA 543613, selectively treated MECP2-KO pathophysiology while leaving controls unaffected.

Nefiracetam is a nootropic agent (i.e., a cholinergic and glutamatergic agonist) that was developed to enhance cognitive functioning. PHA 543613 is an α7-nAChR agonist with proven neuroprotective effects in a neurodevelopmental disease model. Cholinergic neuromodulatory effects are many because nAChRs are widely dispersed across the neuronal and synaptic architecture, and acetylcholine additionally affects the release of other neurotransmitters. The compounds identified in the human MECP2-KO models disclosed herein, encourages their use as treatment options for patients that have abnormal MECP2 expression (e.g., loss-of-function mutations in the MECP2 gene), such as RTT.

A “nootropic agent” as used herein, refers to agents that enhance GABAergic, cholinergic, and/or monoaminergic neuronal systems. In a further embodiment, the nootropic agent is a racetam-based nootropic agent. Racetams are a class of drugs that share a pyrrolidone nucleus. Some of racetams are considered as nootropic agents. Examples of such agents, include but are not limited to, nefiracetam, piracetam, aniracetam, oxiracetam, pramiracetam and phenylpiracetam. In a further embodiment, the racetam-based nootropic agent is nefiracetam:

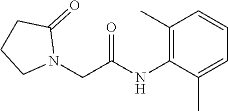 [SMILES: Cc1cccc(C)c1NC(=O)CN1CCCC1=O]

Nefiracetam is a nootropic agent that has shown to possess certain antidementia properties in animals. Nefiracetam's therapeutic actions are mediated by enhancement of GABAergic, cholinergic, and monoaminergic neuronal systems. Nefiracetam shows high affinity for the GABA_{A} receptor (IC₅₀=8.5 nM), where it is presumed to be an agonist. Studies of long-term consumption of nefiracetam in humans and primates have shown it to have no toxicity. Additionally, there has been no evidence of toxicity during clinical trials.

An “alpha-7 nicotinic acetylcholine receptor agonist” or “α7-nAChR agonist” refers to an agent that agonizes alpha-7 nicotinic acetylcholine receptors in the brain. The alphα7 nicotinic acetylcholine receptor is an important part of the cholinergic nerve system in the brain. Moreover, it is associated with a cholinergic anti-inflammatory pathway in the termination of the parasympathetic nervous system. α7-nAChR agonists have shown to have positive effects on neurocognition in persons with schizophrenia. Examples of α7-nAChR agonists include, but are not limited to, N-[(3R)-1-azabicyclo[2.2.2]oct-3-yl]furo[2,3-c]pyridine-5-carboxamide (PHA-543613), bradanicline, encenicline, N-[(3R,5R)-1-azabicyclo[3.2.1]oct-3-yl]furo[2,3-c]pyridine-5-carboxamide (PHA-709829), (4-bromophenyl) 1,4-diazabicyclo[3.2.2]nonane-4-carboxylate (SSR-180), tropisetron, N-[(3S)-1-azabicyclo[2.2.2]oct-3-yl]-1H-indazole-3-carboxamide hydrochloride (RG3487), 1-[6-(4-fluorophenyl)pyridin-3-yl]-3-(4-piperidin-1-ylbutyl) urea (SEN34625/WYE-103914), N-[4-(3-pyridinyl)phenyl]-4-morpholinepentanamide (SEN12333), 5-(6-[(3R)-1-azabicyclo[2,2,2]oct-3-yloxy]pyridazin-3-yl)-1H-indole (ABT-107), clozapine, 3-(2,4-dimethoxy-benzylidene)anabaseine (GTS-21), semapimod, and (2S)-2′H-spiro[4-azabicyclo[2.2.2]octane-2,5′-[1,3]oxazolidin]-2′-one (AR-R17779). In a particular embodiment, the α7-nAChR agonist is PHA-543613:

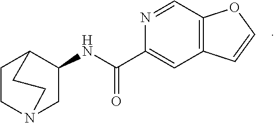 [SMILES: O=C(N[C@H]1CN2CCC1CC2)c1cc2ccoc2cn1]

PHA-543613 is a drug that acts as a potent and selective agonist for the α7 subtype of neural nicotinic acetylcholine receptors, with a high level of brain penetration and good oral bioavailability. It is under development as a possible treatment for cognitive deficits in schizophrenia.

The disclosure provides methods for treating a subject with an X-linked neuronal disorder, or abnormal MeCP2 expression comprising administering a therapeutically effective amount of a nootropic agent and/or α7-nAChR agonist disclosed herein. A therapeutically effective amount can be measured as the amount sufficient to ameliorating or abrogating symptoms associated with an X-linked disorder, or abnormal MeCP2 expression. Generally, the optimal dosage of therapeutic compound(s) will depend upon the type and stage of the cancer and factors such as the weight, sex, and condition of the subject. Nonetheless, suitable dosages can readily be determined by one skilled in the art. Typically, dosages used in vitro may provide useful guidance in the amounts useful for in situ administration of the pharmaceutical composition, and animal models may be used to determine effective dosages for treatment of specific infections. Various considerations are described, e.g., in Langer, Science, 249:1527, (1990); Gilman et al. (eds.) (1990), each of which is herein incorporated by reference. Typically, a suitable dosage for treating an X-linked disorder, or abnormal MeCP2 expression with the therapeutic compound(s) is from 1 mg/kg to 1000 mg/kg body weight, e.g., 150 to 500 mg/kg body weight. In a particular embodiment, a therapeutic compound disclosed herein is administered at dosage of 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg, or a range that includes or is between any two of the foregoing dosages, including fractional dosages thereof.

In a certain embodiment, the disclosure provides a method for treating or rescuing synaptic defects caused by abnormal MeCP2 expression in a subject in need thereof, comprising administering to the subject therapeutically effective amount(s) of a nootropic agent and/or α7-nAChR agonist of the disclosure. In a further embodiment, the abnormal MeCP2 expression is caused by a genetic disease or disorder that affects the expression of the MECP2 gene. Examples of such genetic disease or disorders includes, but is not limited to, Rett syndrome, MECP2 duplication syndrome, neonatal encephalopathy, systemic lupus erythematosus (SLE), and autism spectrum disorder (ASD). In a further embodiment, pharmaceutical compositions comprise a nootropic agent and/or an alpha-7 nicotinic acetylcholine receptor (α7-nAChR) agonist as an active agent in the pharmaceutical composition. In a further embodiment, the pharmaceutical composition comprises pharmaceutically acceptable carriers, diluents, and/or excipients.

In a further embodiment, the disclosure provides a method for treating or rescuing synaptic defects caused by abnormal MeCP2 expression in a subject in need thereof, comprising administering to the subject therapeutically effective amounts of a nootropic agent and an α7-nAChR agonist. In another embodiment, the nootropic agent and the α7-nAChR agonist are administered sequentially. In an alternate embodiment, the nootropic agent and the α7-nAChR agonist are administered concurrently. In a further embodiment, the disclosure provides for a pharmaceutical composition that is formulated for oral delivery which comprises a nootropic agent and an α7-nAChR agonist. In yet a further embodiment, the nootropic agent and/or α7-nAChR agonist is combined with treatments for Rett syndrome or Autism spectrum disorder (ASD).

Further provided herein, are studies which employ an innovative human-based neural model of cellular mosaicism. The model has clear utility for MECP2 and other X-linked genetic disorders, and the concept can likewise be expanded to include other aspects of pharmacogenomic precision medicine, such as characterizing pharmacologic response or variation in cytotoxicity for patients with novel mutations. Furthermore, the impact of somatic mosaicism on clinical and neurodevelopmental phenotypes is becoming increasingly appreciated, so the model developed herein will offer further utility for the study of somatic neuronal mosaicism in other neuropsychiatric diseases and human neurodevelopment in general.

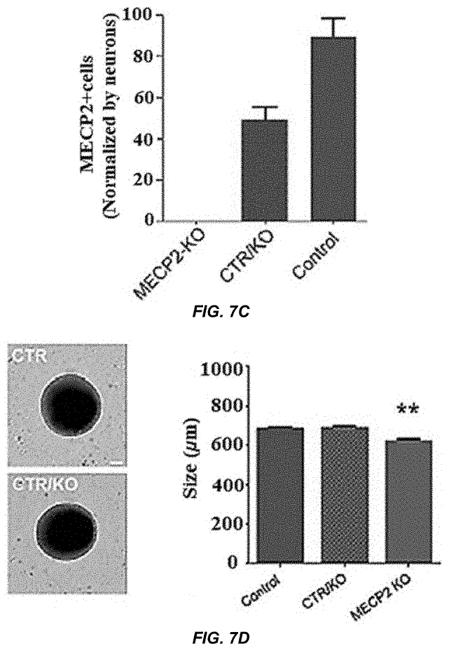

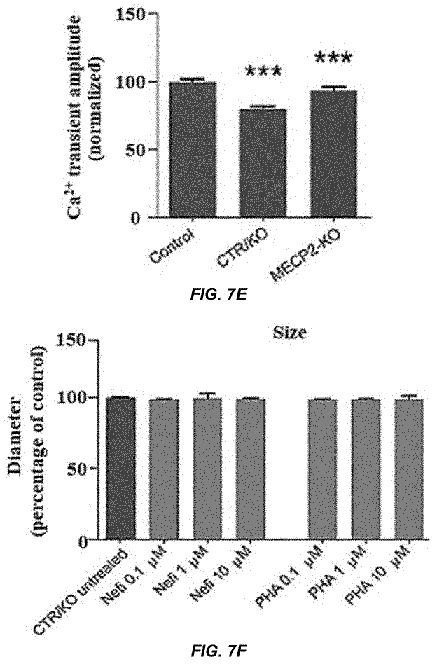

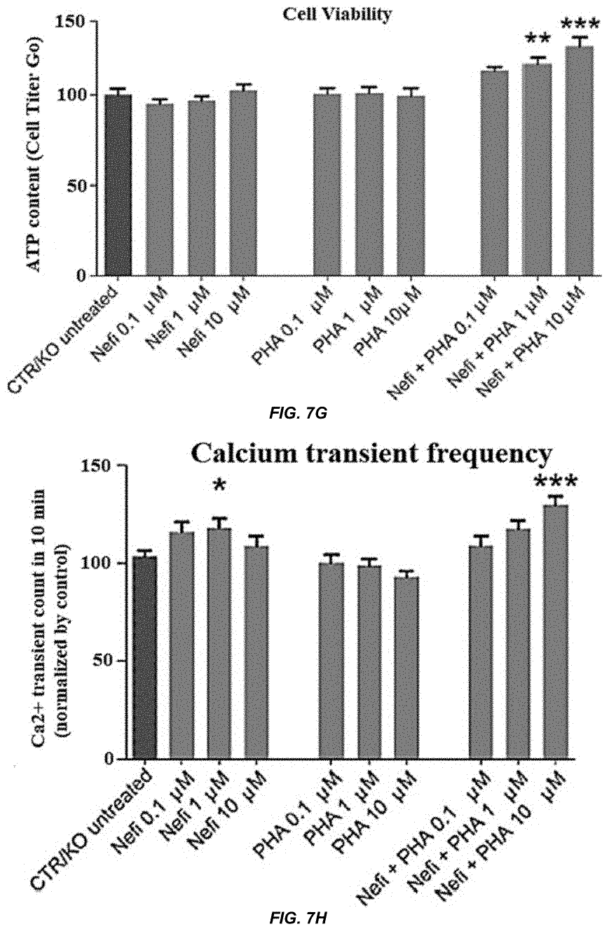

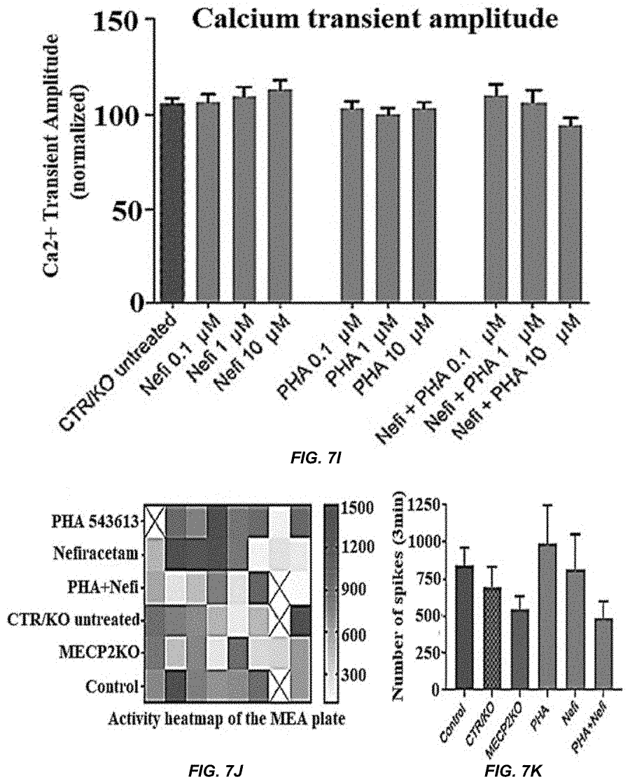

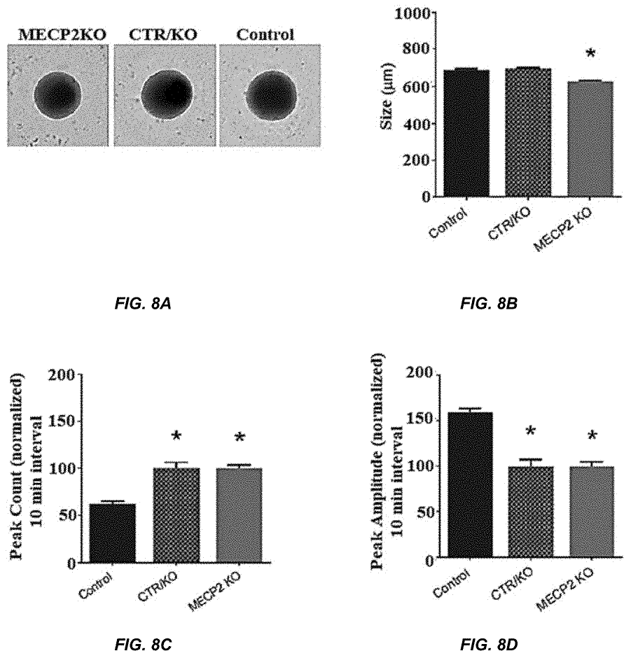

The disclosure further also provides a neural drug screening platform for identifying therapeutic compounds that can ameliorate or rescue deleterious biological effect(s) resulting from abnormal expression of a gene from a chromosome (e.g., X chromosome), comprising: contacting set(s) of neurons that have been differentiated from stem cells (e.g., mammalian stem cells) with a candidate drug, wherein a first set of neurons have been differentiated from stem cells (i.e., induced pluripotent stem cells, embryonic stem cells) that have mutation(s) affecting the normal expression of a gene from the chromosome and optionally, a second set of neurons that are differentiated from stem cells that do not have said mutation(s); (b) contacting set(s) of neurospheres that have been derived from stem cells (e.g., mammalian stem cells) with the candidate drug, wherein a first set of neurospheres comprises neurospheres that have been differentiated from the stem cells that have mutation(s) affecting the normal expression of the gene from an X-chromosome, and optionally, a second set of neurospheres that have been differentiated from stem cells that do not have said mutation(s); and/or (c) contacting set(s) of cortical organoids that have been generated from stem cells with the candidate drug, wherein a first set of cortical organoids have been differentiated from stem cells that have mutation(s) affecting the normal expression of the gene from an X-chromosome, and optionally, a second set of cortical organoids that have been differentiated from stem cells that do not have said mutation(s); (d) evaluating whether the candidate drug rescues or ameliorates rescue deleterious biological effect(s) resulting from abnormal expression of a gene from an X-chromosome in the first set of neurons, and/or the first set of neurospheres, and/or the first set of cortical organoids. In yet another embodiment, the neural drug screening platform, further comprises determining whether the candidate drug is toxic or negatively effects biological activity in neurons, neurospheres, and/or cortical organoids that do not have mutation(s) in the gene being studied. The mutations in the gene can be nonsense mutations, frameshift mutations, loss of function point mutations, etc. The genes to be studied are generally directed to genes that lead to proper neural development and activity. In a particular embodiment, the neural genes to be screened are found on the X-chromosome, i.e., X-linked neural disorders. Examples of such X-linked neural disorders include mutations in genes including, but are not limited to, MECP2, NLGN3, NLGN4, lysosomal alpha-galactosidase A, KDM6A, RPS6KA3, ABCD1, ATP7A, L1CAM, ATRX, and PHF8. In a particular embodiment, the gene to be screened is MECP2, which is associated with Rett Syndrome. As the screening methods are directed to neural cells, the biological activity to be evaluated by the candidate drug can induce calcium oscillations, synaptic activity, and/or synaptic morphology in neurons, neural-like cells or organoids. Moreover, as shown in FIG. 7A, mosaic neurospheres and other neural cell aggregations can be generated by differentiating from stem cells that have mutations in neural genes, and from stem cells that do not. The level of mosaicism can be controlled based upon the ratio of “normal” stem cells to “mutated” stem cells being differentiated into the neurospheres or other neural cell aggregation.

The findings presented herein, indicate the use of nootropic agents and/or α7-nAChR agonist (e.g., Nefiracetam and/or PHA 543613) for the treatment of RTT and other disorders which have an MeCP2 deficiency. The drug-screening platform developed herein was designed to facilitate clinical translatability. Drugs were eliminated that significantly affected controls, thereby limiting clinical error or adverse clinical effects. By using a series of increasingly complex human models with the final, cortical organoids, closely recapitulating in vivo human fetal neurodevelopment, therapeutic agents, such as nefiracetam and PHA 543613, were identified which could reverse the synaptic effects cause by an MeCP2 deficiency. Neurodevelopmental disorders caused by MeCP2 deficiency are severe and treatment options are limited, or altogether unavailable, so the findings presented herein are especially valuable clinically. Namely, nootropic agents and/or α7-nAChR agonist such as Nefiracetam and PHA 543613 improved synaptic morphology, genetic expression, and network activity. Thus, nootropic agents and/or α7-nAChR agonist such as Nefiracetam and/or PHA 543613 can be used as viable treatment option(s) for these difficult to treat patients.

The disclosure further provides for a pharmaceutical composition comprising therapeutic compound(s) comprising nootropic agents and/or α7-nAChR agonist disclosed herein. Any of a variety of art-known methods can be used to administer a therapeutic compound(s) comprising nootropic agents and/or α7-nAChR agonist disclosed herein. For example, administration can be parenterally, by injection or by gradual infusion over time. The therapeutic compound(s) alone or with the other therapeutic agents can be administered orally, intravenously, intraperitoneally, intramuscularly, subcutaneously, intracavity, by inhalation, or transdermally.

A pharmaceutical composition comprising therapeutic compound(s) of the disclosure can be in a form suitable for administration to a subject using carriers, excipients, diluents and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15^{th} ed., Easton: Mack Publishing Co., 1405-1412, 1461-1487 (1975), and The National Formulary XIV., 14^{th} ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7^{th} ed.).

The disclosure further provides for a pharmaceutical composition comprising nootropic agents and/or α7-nAChR agonist therapeutic compound(s) disclosed herein that can be administered in a convenient manner, such as by injection (subcutaneous, intravenous, etc.), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

A “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the individual's diet. For oral therapeutic administration, the pharmaceutical composition can be incorporated with excipients and used in the form of ingestible tablets, buccal tablets, troches, capsules, elixirs, suspensions, syrups, wafers, chewable tablets, gummies, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic/biocompatible in the amounts employed. In addition, the pharmaceutical composition can be incorporated into sustained-release preparations and formulations.

In further embodiments, the disclosure further provides that oral pharmaceutical formulations comprising therapeutic compound(s) disclosed herein may have an enteric coating. As used herein “enteric coating”, is a material, a polymer material or materials which encase the medicament core (e.g., prodrug the disclosure). Typically, a substantial amount or all of the enteric coating material is dissolved before the medicament or therapeutically active agent is released from the dosage form, so as to achieve delayed dissolution of the medicament core. A suitable pH-sensitive polymer is one which will dissolve in intestinal juices at a higher pH level (pH greater than 6), such as within the small intestine and therefore permit release of the pharmacologically active substance in the regions of the small intestine and not in the upper portion of the GI tract, such as the stomach. An “enterically coated” drug or tablet refers to a drug or tablet that is coated with a substance—i.e., with an “enteric coating”—that remains intact in the stomach but dissolves and releases the drug once the small intestine is reached.

The coating material is selected such that the therapeutically active agent will be released when the dosage form reaches the small intestine or a region in which the pH is greater than pH 6. The coating may be a pH-sensitive material, which remains intact in the lower pH environs of the stomach, but which disintegrate or dissolve at a more neutral pH commonly found in the small intestine of the patient. For example, the enteric coating material begins to dissolve in an aqueous solution at pH between about 6 to about 7.4. For example, pH-sensitive materials will not undergo significant dissolution until the dosage form has emptied from the stomach and proximal portions of the small intestine. The pH of the small intestine gradually increases to about 6.5 in the duodenal bulb to about 7.2 in the distal portions of the small intestine (ileum).

Enteric coatings have been used for many years to arrest the release of the drug from orally ingestible dosage forms. Depending upon the composition and/or thickness, the enteric coatings are resistant to stomach acid for required periods of time before they begin to disintegrate and permit release of the drug in the lower stomach or upper part of the small intestines. Examples of some enteric coatings are disclosed in U.S. Pat. No. 5,225,202 which is incorporated by reference fully herein. As set forth in U.S. Pat. No. 5,225,202, some examples of coating previously employed are beeswax and glyceryl monostearate; beeswax, shellac and cellulose; and cetyl alcohol, mastic and shellac, as well as shellac and stearic acid (U.S. Pat. No. 2,809,918); polyvinyl acetate and ethyl cellulose (U.S. Pat. No. 3,835,221); and neutral copolymer of polymethacrylic acid esters (Eudragit L30D) (F. W. Goodhart et al., Pharm. Tech., pp. 64-71, April 1984); copolymers of methacrylic acid and methacrylic acid methylester (Eudragits), or a neutral copolymer of polymethacrylic acid esters containing metallic stearates (Mehta et al., U.S. Pat. Nos. 4,728,512 and 4,794,001). Such coatings comprise mixtures of fats and fatty acids, shellac and shellac derivatives and the cellulose acid phthalates, e.g., those having a free carboxyl content. See, Remington's at page 1590, and Zeitova et al. (U.S. Pat. No. 4,432,966), for descriptions of suitable enteric coating compositions.

Generally, the enteric coating comprises a polymeric material that prevents prodrug release in the low pH and mildly acidic environment of the stomach and proximal small intestine but that ionizes at a higher pH, typically at a pH of 6 or higher, and thus dissolves sufficiently in the mid to lower small intestines to gradually release the active agent therein. Accordingly, among the most effective enteric coating materials are polyacids having a pKₐ in the range of about 3 to 6. Suitable enteric coating materials include, but are not limited to, polymerized gelatin, shellac, methacrylic acid copolymer type C NF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), and acrylic acid polymers and copolymers, typically formed from methyl acrylate, ethyl acrylate, methyl methacrylate and/or ethyl methacrylate with copolymers of acrylic and methacrylic acid esters (Eudragit NE, Eudragit RL, Eudragit RS). For example, the enterically coating can comprise Eudragit L30D, triethylcitrate, and hydroxypropylmethylcellulose (HPMC), Cystagon® (or other cysteamine derivative), wherein the coating comprises 10 to 13% of the final product.

In another embodiment, the therapeutic composition can be formulated as an immediate release formulation such that it is delivered in the upper gastrointestinal tract. In still another formulation the therapeutic composition can comprise a gastroretentive formulation comprising, e.g., carbopol or some other hydrophilic polymer.

Preparations for parenteral administration of a composition comprising therapeutic compound(s) of the disclosure include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, anti-oxidants, cheating agents, inert gases and the like also can be included.

Pharmaceutical compositions suitable for injectable use include sterile aqueous solutions (where water soluble) or dispersions and sterile powders for the extemporaneous preparation of sterile injectable solutions or dispersions. In all cases, the composition should be sterile and should be fluid to the extent that easy syringability exists. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be typical to include isotonic agents, for example, sugars, polyalcohols, such as mannitol, sorbitol, or sodium chloride in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

It is especially advantageous to formulate parenteral compositions in dosage unit form for ease of administration and uniformity of dosage. “Dosage unit form” as used herein, refers to physically discrete units suited as unitary dosages for the individual to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieve.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

The pharmaceutical compositions according to the disclosure may be administered at a therapeutically effective amount either locally or systemically. As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of infection in a subject, the age, sex, and weight of the individual. Dosage regime can be adjusted to provide the optimum therapeutic response. For example, several divided doses can be administered daily or the dose can be proportionally reduced as indicated by the exigencies of the therapeutic situation.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more therapeutic compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise therapeutic compounds disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### EXAMPLES

Generation of MECP2-KP cell lines and cell culture. Control PSCs were characterized as previously described in Nageshappa et al. (Mol. Psychiatry 21:178-188 (2016)). MeCP2-deficient cell lines were generated via pluripotent induction of male patient-derived fibroblasts, and a frameshift mutation was CRISPR/Cas9-edited into the H9 hESC MECP2 locus, creating an early stop codon and non-functional MeCP2. Exome sequencing confirmed mutagenesis and analyzed off-targets. PSC colonies were maintained on Matrigel-coated dishes (BD-Biosciences, San Jose, Calif., USA) and fed daily with mTeSR1 (StemCell Technologies, Vancouver, Canada); only mycoplasma-negative cell cultures were used. Cells were evaluated by karyotype and CNV arrays to detect genomic alteration.

Karyotyping: G-banding karyotyping analyses were performed by Molecular Diagnostics Service (San Diego, Calif., USA).

Neuronal differentiation of iPSCs. Differentiation was as performed as described by Chailangkam et al. (Nature 536:338-343 (2016)). mTeSR1 used to feed iPSCs was replaced after 7 days(d) with N2 medium [DMEM/F12 (Life Technologies, Carlsbad, Calif., USA), 0.5×N2 (Life Technologies), 1 μM Dorsomorphin (Dorso; R&D Systems, Minneapolis, Minn., USA), and 1 μM SB431542 (SB; Stemgent, Cambridge, Mass., USA)], and embryoid bodies (EBs) were formed. Dissociated EBs were plated and fed N2B27 medium [N2 medium with 0.5×B27-Supplement (Life Technologies), 1% penicillin/streptomycin (PS; Life Technologies), and 20 ng/mL FGF-2]. Dissociated rosettes were poly-ornithine/laminin-plated . Emergent NPCs were fed N2B27 on alternate days; withdrawal of FGF-2 induced neuronal differentiation.

Generation of cortical organoids. Cortical organoid generation was performed according to Trujillo et al. (Cell Stem Cell 25:558-569.e7 (2019)). iPSCs cultured 5-7 d were dissociated with 1:1 Accutase (Life Technologies) in PBS, and 4×10⁶ cells/well in mTeSR1 supplemented with 10 μM SB (Stemgent), 1 μM Dorso (R&D Systems), and 5 μM Y-27632 (EMD-Millipore, Burlington, Mass., USA) were plated on rotating suspension. Formed spheres were fed mTeSR1 (with 10 μM SB, 1 μM Dorso)×3 d followed by: Medial [Neurobasal (Life Technologies), 1× Glutamax (Life Technologies), 2% Gem21-NeuroPlex (Gemini Bio-Products, Sacramento, Calif., USA), 1% N2-NeuroPlex (Gemini Bio-Products), 1% non-essential AAs (NEAA; Life Technologies), 1% PS (Life Technologies), 10 μM SB, and 1 μM Dorso]×7 d; Media2 (Neurobasal, 1× Glutamax, 2% Gem21, 1% NEAA, and 1% PS) with 20 ng/mL FGF2 (Life Technologies)×7 d; Media2 with 20 ng/mL each of FGF2 and EGF (PeproTech, Rocky Hill, N.J., USA)×7 d; and Media2 with 10 ng/mL each of BDNF, GDNF, and NT-3 (all PeproTech), 200 μM L-ascorbic acid (Sigma-Aldrich, St. Louis, Mo., USA), and 1 mM dibutyryl-cAMP (Sigma-Aldrich)×7 d. Cortical organoids were subsequently maintained indefinitely in Media2 changed every 3-4 days.

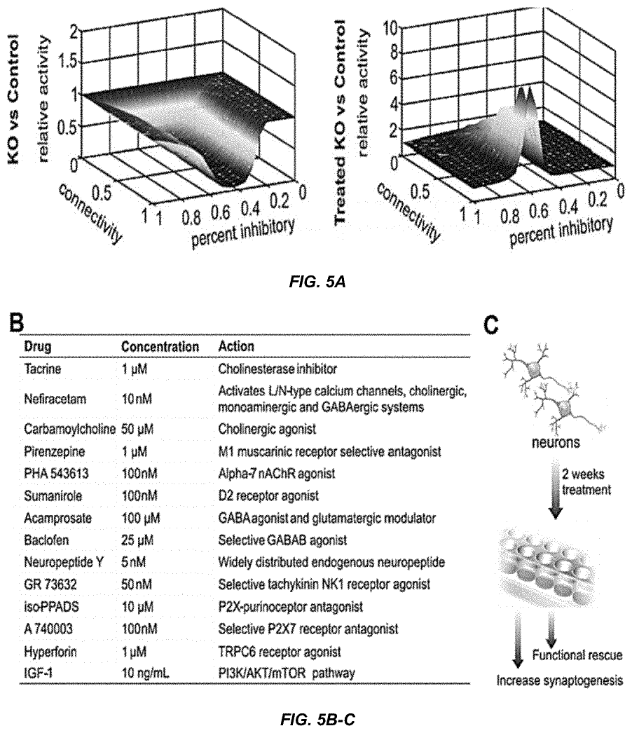

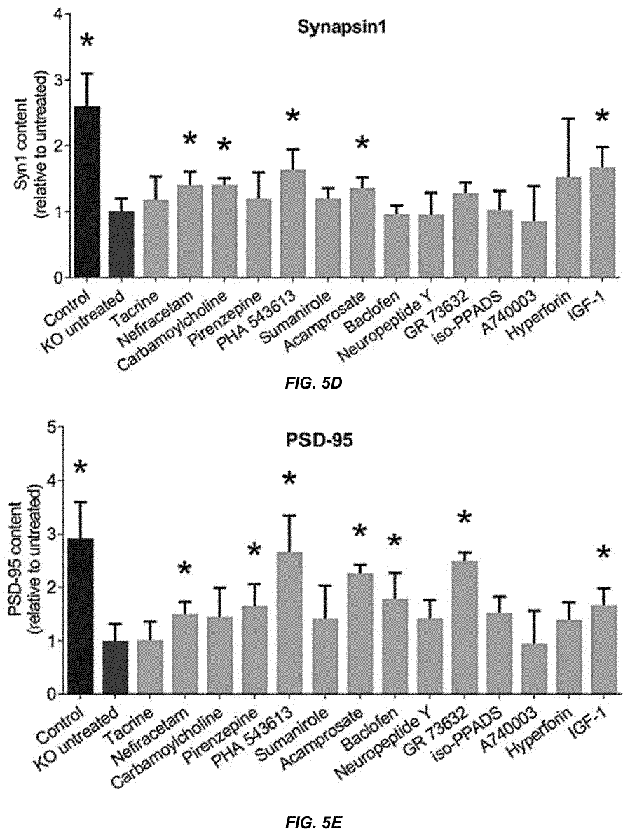

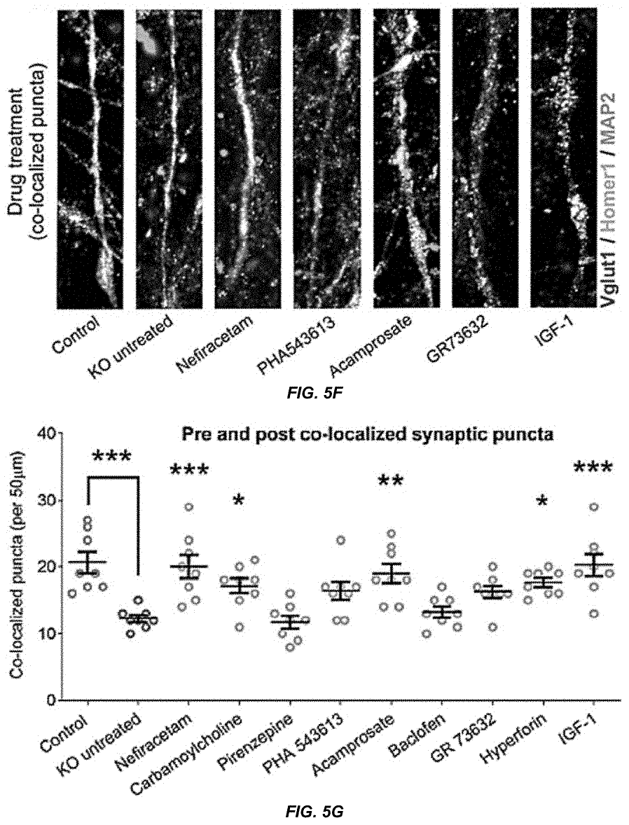

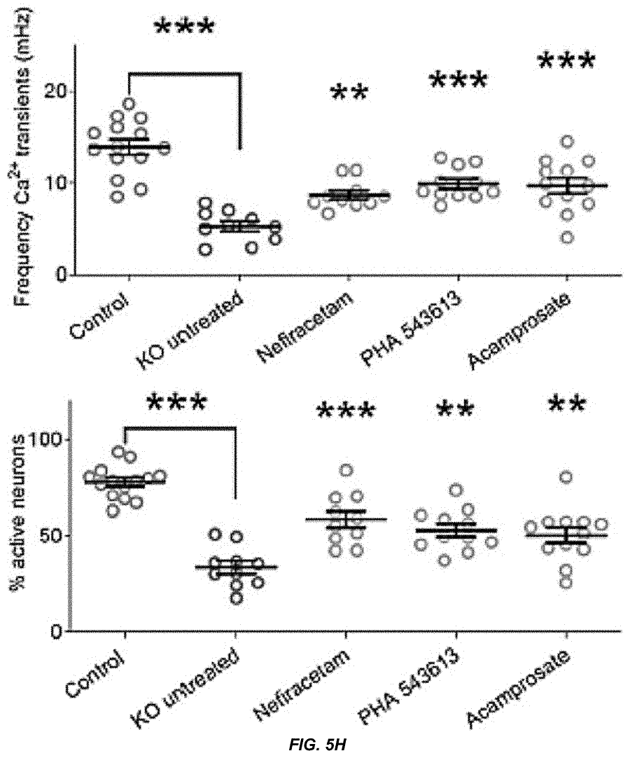

Pharmacologic treatment. One-month old neurons were treated for two weeks at the drug concentrations listed in FIG. 5B. One-month old cortical organoids were treated for 1 mo with either Nefiracetam (10 μM) or PHA 543613 (10 μM) during media changes.

RNA extraction and PCR Array. TRIzol reagent (Life Technologies) extracted total RNA, and TURBO DNase (Life Technologies) removed contaminating DNA (each per manufacturer's instructions). DNase-treated RNA was assessed by NanoDrop1000 (ThermoScientific, Waltham, Mass., USA). Total RNA was converted to cDNA and evaluated RT PCR Array per manufacturer's instructions (Qiagen, Hilden, Germany).

Single-cell qRT-PCR. Single-Cell and BioMark HD Systems (Fluidigm, San Francisco, Calif., USA) were used for specific target amplification in NPCs, neurons, and organoids as described in Trujillo et al. Briefly, single cells (following dissociation, for organoids) were captured on a Cl chip, and a LIVE/DEAD Cell Viability/Cytotoxicity kit (Life Technologies) assessed viability. DELTAgene primer pairs (96.96 Dynamic Array IFC chip) were used for single-cell qPCR; results analysis by Fluidigm Real-time PCR Analysis Software and Singular Analysis Toolset 3.0.

RNA-seq analyses. RNA-seq analyses were performed as described in Marchetto et al. (Nature 504:525-529 (2013)). Briefly, RNeasy Mini kit (Qiagen) isolated RNA for library preparation (Illumina TruSeq RNA Sample Preparation Kit; San Diego, Calif., USA) and sequencing (Illumina HiSeq2000, 50 bp paired-end reads, 50 million high-quality sequencing fragments per sample on average).

Gene ontology (GO) analysis. RNA-seq enrichment was with plugins WebGestalt and Cytoscape and considered only statistically significant categories (P<0.05). Genes evaluated for differential expression set the background for GO annotation and enrichment analysis.

Immunofluorescence of cells and cortical organoids. Immunofluorescence was performed as described in Chailangkam et al. Cells fixed in 4% PFA were PBS-washed and permeabilized/blocked (0.1% Triton X-100, 2% BSA). Cortical organoids fixed in 4% PFA were transferred to 30% sucrose, sunken, embedded in O.C.T. (Sakura, Tokyo, Japan), and cryostat-sectioned. Air-dried slides with organoid sections were permeabilized/blocked (0.1% Triton X-100, 3% FBS, PBS). Primary antibodies (goat anti-Nanog, Abcam (Cambridge, UK) ab77095, 1:500; rabbit anti-Lin28, Abcam ab46020, 1:500; mouse anti-MeCP2, Sigma-Aldrich M7443, 1:500; rabbit anti-Oct4, Abcam ab19857, 1:500; mouse anti-Nestin, Abcam ab22035, 1:200(organoid: 1:250); rat anti-CTIP2, Abcam ab18465, 1:250(organoid: 1:500); rabbit anti-SATB2, Abcam ab34735, 1:200; chicken anti-MAP2, Abcam ab5392, 1:1000(organoid: 1:2000); rabbit anti-Synapsin1, EMD-Millipore AB1543P, 1:500; mouse anti-Vglut1, Synaptic Systems (Goettingen, Germany) 135311, 1:500; rabbit anti-Homer1, Synaptic Systems 160003, 1:500; mouse anti-NeuN, EMD-Millipore MAB377, 1:500; rabbit anti-Ki67, Abcam ab15580, 1:1000; rabbit anti-SOX2, Cell Signaling Technology (Danvers, Mass., USA) 2748, 1:500; rabbit anti-GFAP, DAKO (Carpinteria, Calif., USA) Z033429, 1:1000; rabbit anti-TBR1, Abcam ab31940, 1:500; rabbit anti-TBR2, Abcam ab23345, 1:500; rabbit anti-beta-catenin, Abcam E247, 1:200; mouse anti-GABA, Abcam ab86186, 1:200; rabbit anti-PROX1, Abcam ab101651, 1:250) incubated overnight at 4° C. Cells or slides were then PBS-washed and incubated with secondary antibodies (Alexa Fluor 488, 555, and 647, Life Technologies, 1:1000). DAPI (1:10000) stained nuclei. Slides or coverslips were mounted with ProLong Gold anti-fade mountant (Life Technologies) and analyzed with a Z1 Axio Observer Apotome fluorescence microscope (Zeiss, Oberkochen, Germany).

Teratoma assay. PSC colonies were dissociated, centrifuged, resuspended in 1:1 Matrigel:PBS, and subcutaneously injected in nude mice. Teratomas were excised, fixed, and sliced after 1-2 months, and sections were stained with H&E.

Artificial neural network simulations. Simulations of neural network activity were done with artificial recurrent neural networks using biologically plausible parameter choices, defined by five free parameters: number of neurons in network (N), connection sparsity or percent connectivity (pcon), percentage of inhibitory vs excitatory neurons (pinh), synaptic weights (W), and synaptic decay time constants of the neurons (τ). A network size of N=512 neurons was used; simulations performed with larger and smaller network sizes yielded qualitatively similar results. Percent connectivity (pcon) refers to the percentage of all possible pairwise connections in the network for which a connection was present (non-zero entry in W). The networks followed Dale's Law, which states that any given neuron is either excitatory or inhibitory: For each simulation, a particular excitatory-inhibitory balance was chosen determining the percent of neurons that were inhibitory (pinh) for the network. The artificial neural network implementation was similar to the continuous rate networks outlined in Kim et al. (PNAS 116:22811-22820 (2019)), where a detailed description of the full model can be found. Briefly, each simulated network consisted of a pool of neurons that could be connected to any other neuron in the pool. Each neuron received weighted input from other neurons, and integrated these inputs to produce a firing rate. The network dynamics are governed by the Eq. 1:

$$τ \frac{dx}{dt} = - x + W r \qquad ( 1 )$$

where τ ∈ [symbol] ^{1×N} the synaptic decay time constant for the N neurons in the network, x ∈ [symbol] ^{1×N} is the synaptic current variable for the N neurons, W ∈ [symbol] ^{N×N} is the matrix of synaptic weights between all N neurons, and r ∈ [symbol] ^{1×N} is the output firing rates of the N neurons in the network. The output firing rate for the neurons is given by an element-wise nonlinear transformation of the synaptic current variable. In the network the standard logistic sigmoid function was implemented by EQ. 2:

$$r = \frac{1}{1 + exp ( - x )} \qquad ( 2 )$$

The synaptic connectivity matrix W was randomly initialized from a normal distribution with zero mean and standard deviation g/\sqrt{N⇄ρ_{con}}, where ρ_{con} is the connection sparsity of the network, and g is the gain term. For the networks, g=15 was used, as previous studies have shown that networks operating in a high gain regime (g≥1.5) support rich dynamics analogous to those of biological networks. The synaptic decay time constants were randomly initialized to a value in the biologically plausible range of 20-100 ms. The first-order Euler approximation method was used to discretize EQ. 1 for the simulations.

To implement different levels of synaptic knock-down or strengthening, a synaptic weight scaling factor ρ_{KD} was introduced. Based on the findings of Marchetto et al. (Cell 143:527-539 (2010)) that synaptic strength was decreased in an iPSC model of Rett syndrome particularly for excitatory neurons, perturbations of synaptic strength was applied to excitatory synaptic connections only. To model synaptic knock-down or strengthening the excitatory synaptic weights were multiplied by the scaling factor, ρ_{KD}. To perform each simulation, a network was generated with randomly initialized synaptic weights and synaptic time constants as described above. The simulation was then run for 200-time steps (1-time step simulates 5 ms). For each combination of parameters [ρ_{con}, ρᵢₙₕ, ρ_{KD}], 100 independent simulations were run and the average of all neurons' firing rates on the last time step was taken from each simulation as a measure of network activity. The average network activity across all 100 simulations was taken as an estimate for the network activity for the given set of parameters. For every [ρ_{con}, ρᵢₙₕ] pair simulations were run for the ρ_{KD}, values of interest as well as a “control” network for which ρ_{KD}=1, i.e., no synaptic scaling was applied. All measures of network activity were normalized to that of the corresponding control network to obtain the relative network activity.

To create the relative activity surface plots the simulations described above was run for all networks with ρ_{con} ∈ {1e−5,0.05,0.1,0.15, . . . 0.95,1.0} and ρᵢₙₕ ∈{0.0,0.05,0.1, . . . 0.95,1.0}, representing 441 [ρ_{con}, ρᵢₙₕ] pairs tiling the possible space of network connectivity and excitatory-inhibitory balance. Simulations across this space were run for each of the synaptic perturbation conditions taken from Marchetto et al.: Rett syndrome-like synaptic knockdown (ρ_{KD}=0.375) IGF1-like synaptic improvement (ρ_{KD}=1.5), and control condition (ρ_{KD}=1) (A minimum value of ρ_{con}=1e−5 was used since ρ_{con} must be greater than zero to calculate the standard deviation of the random initialization distribution for the synaptic weights as described above.

Western blotting. Total protein (20 μg) was extracted via Bolt 4-12% Bis-Tris Plus Gel (Life Technologies) and transferred to a nitrocellulose membrane via iBlot2 dry blotting system (ThermoScientific). Membranes were blocked (Rockland Immunochemicals, VWR International, Arlington Heights, Ill., USA); primary antibodies incubated overnight at 4° C. and secondary antibodies for 1 h at ambient temperature. Proteins were detected with an Odyssey CLx infrared imaging system (LI-COR Biosciences, Lincoln, Nebr., USA).

Synaptic puncta quantification. Co-localized pre and postsynaptic puncta (Vglut and Homer1 immunostained) along MAP2-positive processes were quantified. Primary antibodies (see above) incubated 2 h, secondary antibodies incubated 1 h, and coverslips were mounted. Slides were analyzed under fluorescence microscopy.

Spine density and morphology/stability/motility. pHIV7/Syn-EGFP lentivirus was prepared by transfecting HEK cells using the transfection agent polyethyleneimine (Polysciences, Inc., Warrington, Pa., USA), pSyn-EGFP and packaging plasmids pMDL, Rev RSV and VSVG. During cortical neuron differentiation, on day 40, control and MECP2dup neurons were transduced with pHIV7/syn-EGFP. Post transduction, neurons were further differentiated until day 70 and were fixed with 4% PFA and further processed for imaging. Confocal images were obtained using a confocal laser-scanning microscope (Nikon), 60× oil objective, with a sequential acquisition setting at 4096×4096 pixels resolution. Each image is a Z-series projection of ˜7 to 15 images, averaged 2 times and taken at 0.5 μm depth intervals. GFP-labeled differentiated neurons were chosen randomly for quantification. Quantitative analysis was performed in a blinded manner. Dendrites on primary branches were selected randomly and for each neuron two to three segments of 10 μm were analyzed. Morphometric measurements were performed manually using the ImageJ software (1.44p version). Each spine on the dendrites was manually traced. The length and head width of each spine was automatically measured and used to categorize the spines. Specifically, spines where defined as thin and filopodia (immature) or mushroom and stubby (mature) according to the criteria defined previously. Twenty-two (51 segments of 10 μm) and 18 (42 segments of 10 μm) neurons were analyzed for the control and MECP2dup neurons, respectively; two clones for each condition.

Analysis of neuronal morphology. PSC-derived neuronal morphometry was performed as described in Chailangkarn et al. Briefly, neurons with branching neurites featuring dendritic spine-like protrusions were selected, and neuronal morphology was quantified using Neurolucida v9 (MBF Bioscience, Williston, Vt.) via Nikon Eclipse E600 microscope (40× oil). Measurements were as follows: Spine number: number of spines protruding from neurites; soma size: soma area on cross-section; spine length: summed length of all spines per neuron. The rater traced the same neuron after a period of time to ensure intra-rater reliability.

Calcium imaging. Calcium imaging was performed as described in Chailangkarn et al. Briefly, PSC-derived neuronal networks were lentivirally transduced with a Syn::RFP reporter construct. Cultures were washed with Krebs HEPES buffer and incubated with Fluo-4AM (Molecular Probes/Invitrogen, Carlsbad, Calif., USA). A Hamamatsu ORCA-ER digital camera (Hamamatsu Photonics, Japan) with 488 nm filter on an Olympus IX81 inverted fluorescence confocal microscope (Olympus Optical, Tokyo, Japan) acquired 5000 frames at 28 Hz in a 256×256 pixel region (100× magnification). Images were acquired with MetaMorph7.7 (MDS Analytical Technologies, Sunnyvale, Calif., USA) and analyzed with ImageJ and Matlab7.2 (Mathworks, Natick, Mass., USA). Syn::RFP+ neurons were selected following tetrodotoxin blockade of calcium transients. A 95th-percentile threshold of amplitude for calcium spikes was set for event detection; signal amplitude is presented as fluorescence change (ΔF/F) following background subtraction.

Multi-electrode array (MEA) analysis. MEA analyses of neurons and cortical organoids were performed as described in Nageshappa et al. (Mol. Psychiatry 21:178-188 (2016)). Briefly, 3D neurosphere cultures of NPC derivation were plated on dual-chamber MEA; spontaneous spike activity was evaluated with the MED64 System (Panasonic). Glutamatergic (AP5, NBQX), GABAergic (Gabazine), and gap channel (Mefloquine) antagonism were used to check neuronal activity. After measurement, neurons were immunostained to check morphology and density. Recorded spikes were analyzed with the software Neuroexplorer (Nex Technologies, Madison, Ala., USA). For cortical organoids, 1-month organoids were plated on MEA plates and recordings were collected with a Maestro MEA system and AxIS Software Spontaneous Neural Configuration (Axion Biosystems, Atlanta, Ga., USA) using a customized script. The plate rested in the machine for three minutes followed by four minutes of recording. Axion Biosystems' Neural Metrics Tool classified electrodes with at least 5 spikes/min as active. Bursts were identified using an inter-spike interval (ISI) threshold requiring a 5-spike minimum and 100 ms maximum ISI. Network bursts required a minimum of 10 spikes under the same ISI and at least 25% active electrodes. The synchrony index was calculated using a 20 ms cross-correlogram synchrony window was used to determine the synchrony index. Bright-field images were captured to assess for cell density and electrode coverage.

Statistical analysis. Statistical analysis throughout was performed using GraphPad Prism (GraphPad Software, La Jolla, Calif.), except for the in silico neural network model described above. Results for continuous variables were expressed as mean±SD, and 95% confidence intervals were normal-based. Means were compared between groups using, where appropriate, unpaired Student's t-test, one-way, or two-way analysis of variance. Tests were performed two-sided with α throughout set as 0.05.

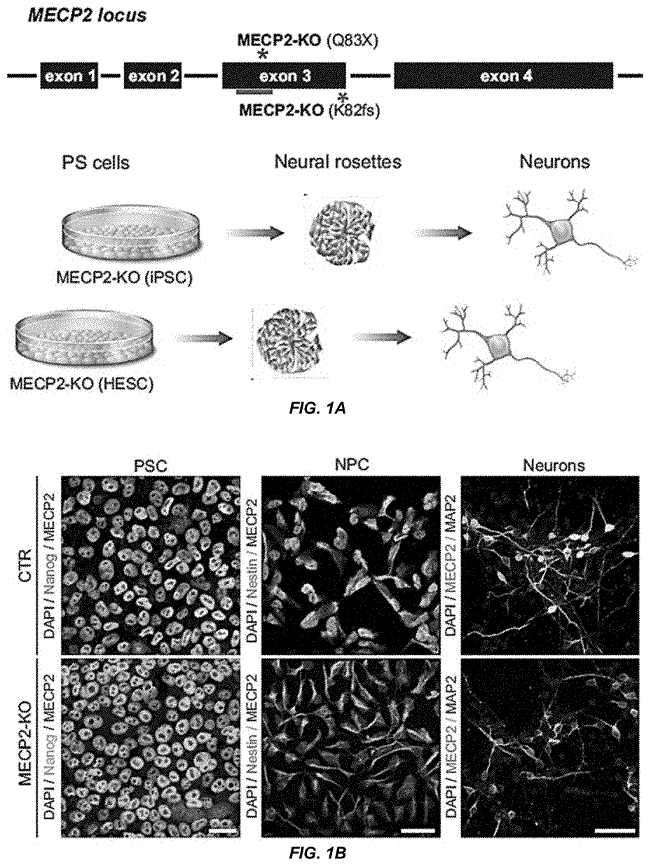

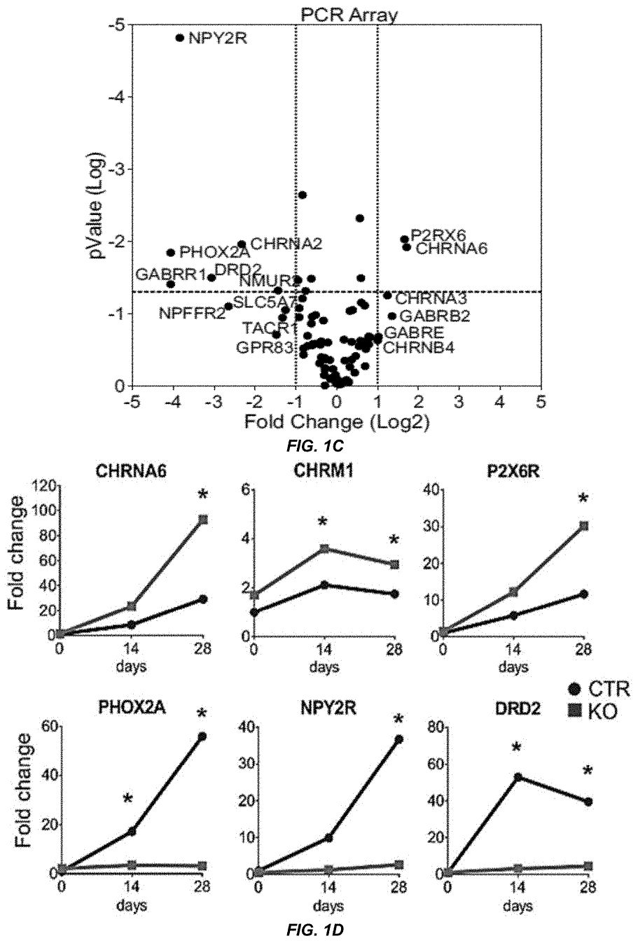

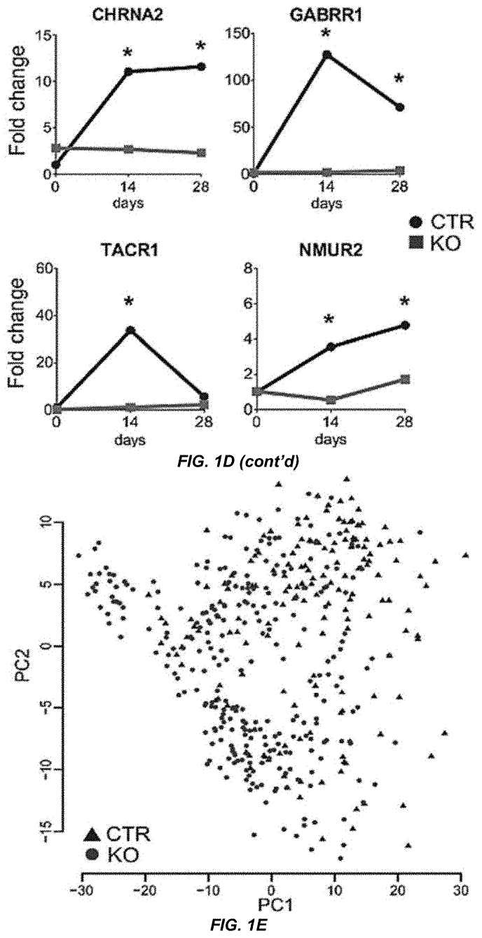

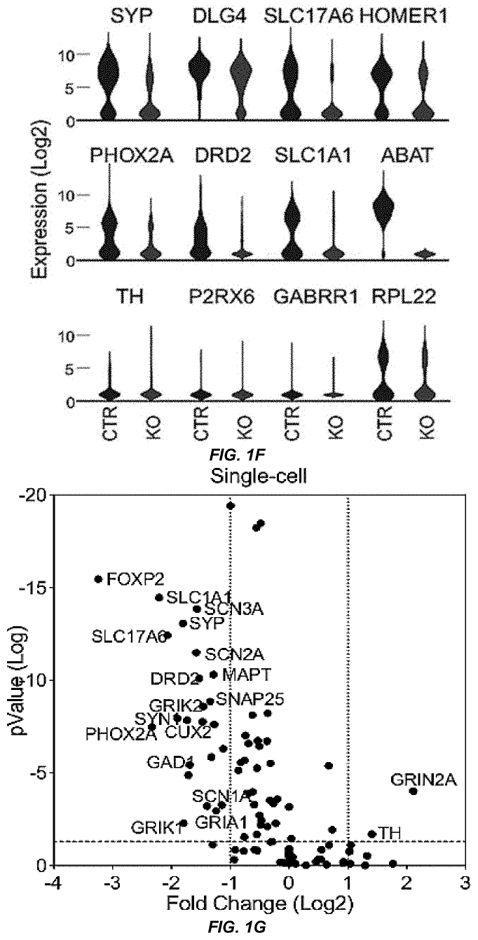

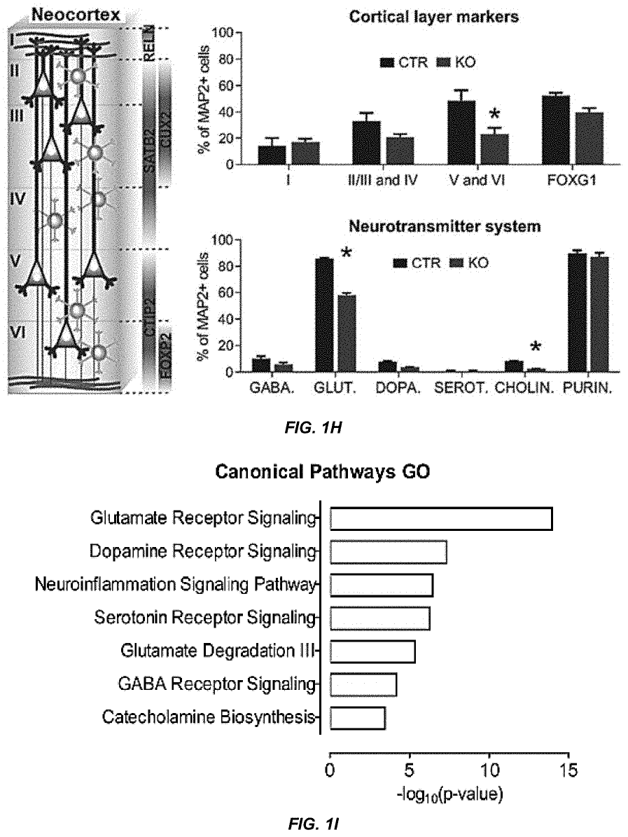

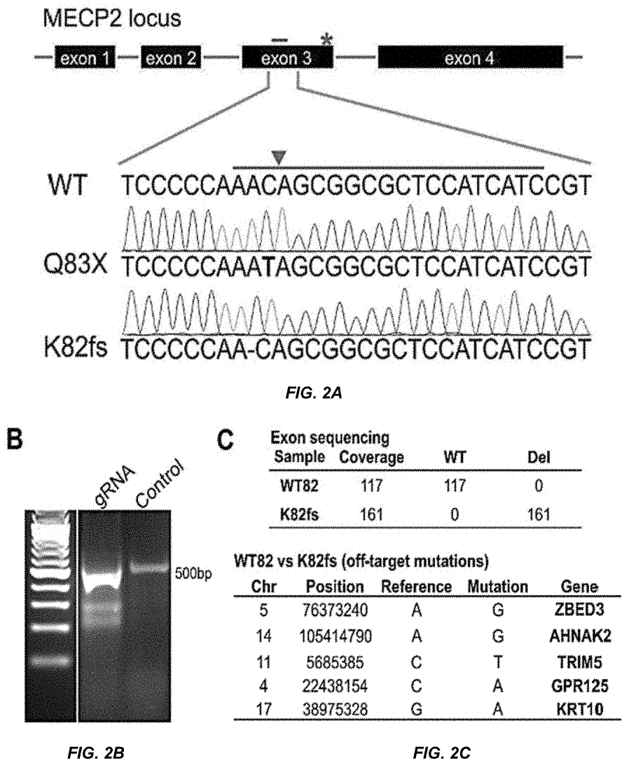

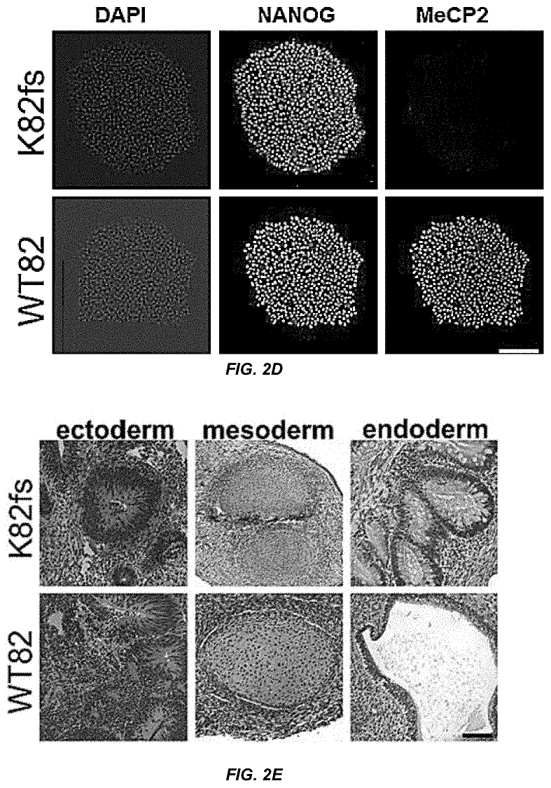

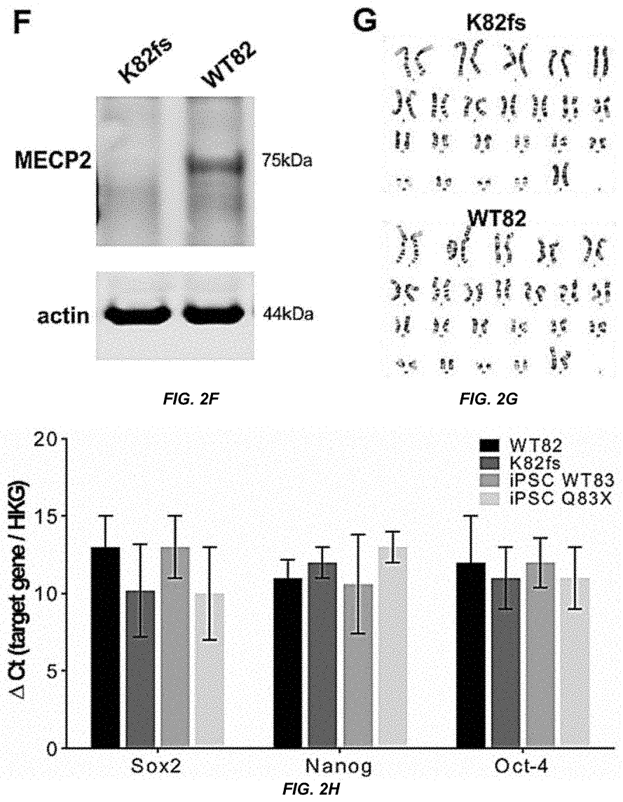

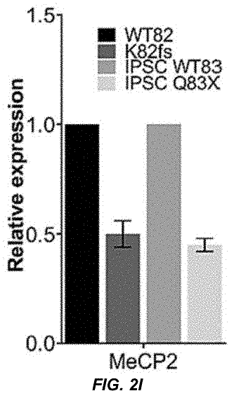

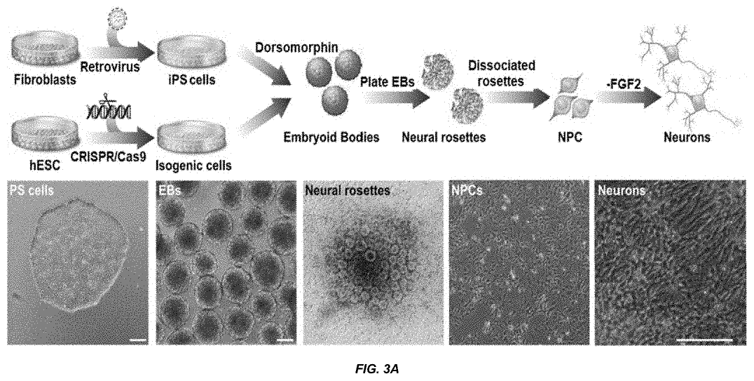

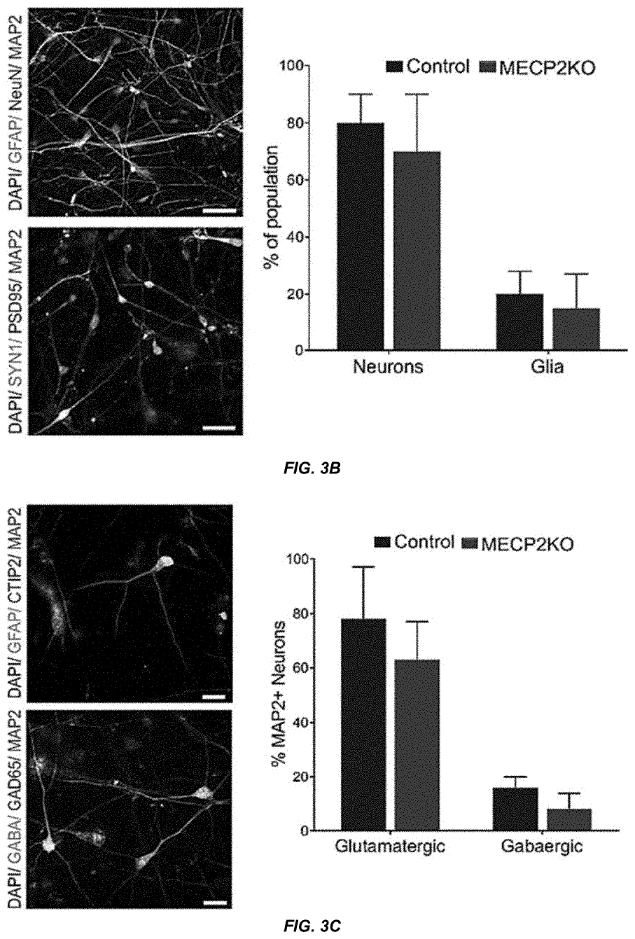

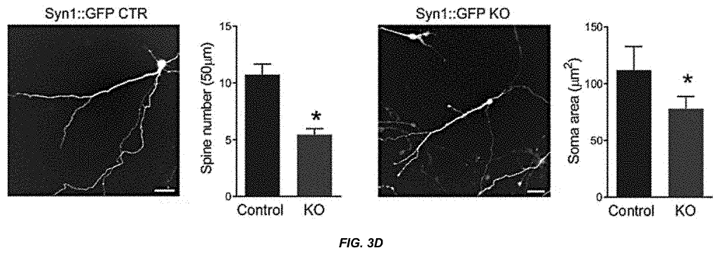

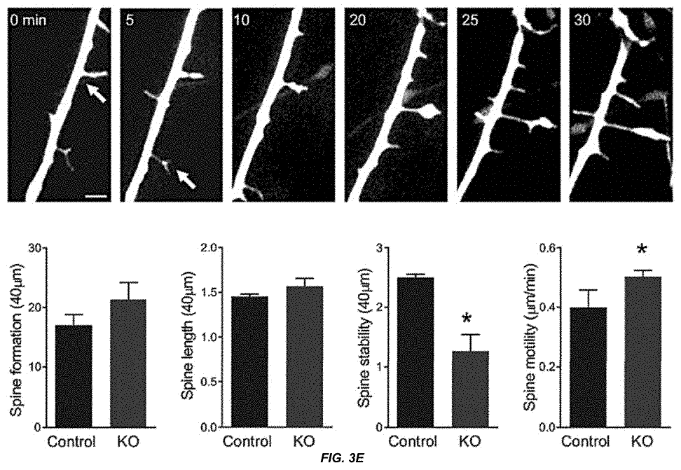

MECP2-KO produces synaptic pathology in human neurons. To develop human models of MeCP2 deficiency, human PSCs with distinct MECP2 exonic loss-of-function mutations (Q83X nonsense or K82 frameshift), isogenic and familial PSC controls, were differentiated into cortical neurons (see FIG. 1A-B, FIG. 2A-C, and FIG. 3A-C). The morphologies of control and MECP2-KO neurons. Compared to controls were first compared. MECP2-KO neurons transfected with Syn1::GFP had decreased soma areas and spine density. Despite no observable difference in either formation or length, the spine-like protrusions in MECP2-KO neurons were less stable and more motile (see FIG. 3D-E), supporting previous descriptions of RTT neuronal pathology.

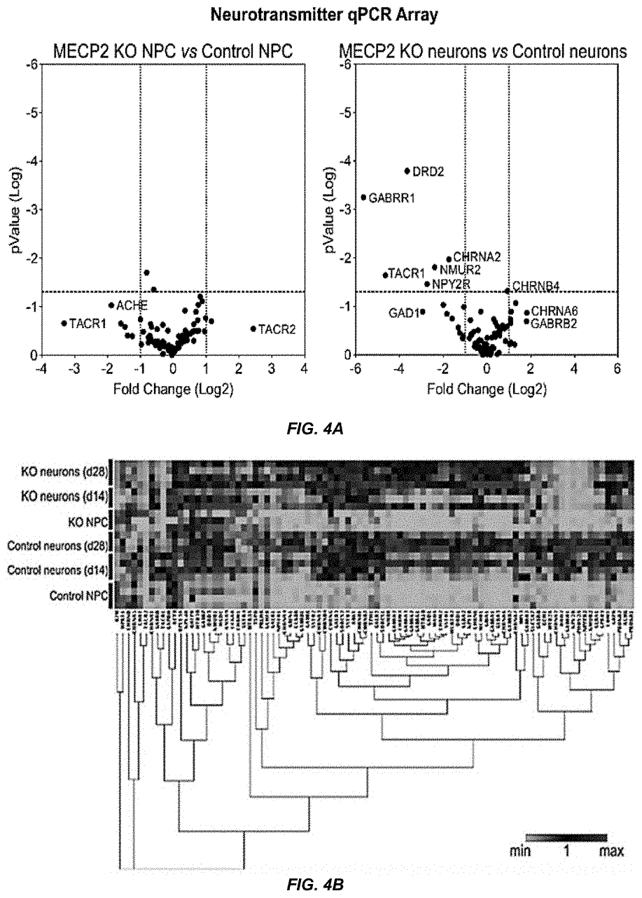

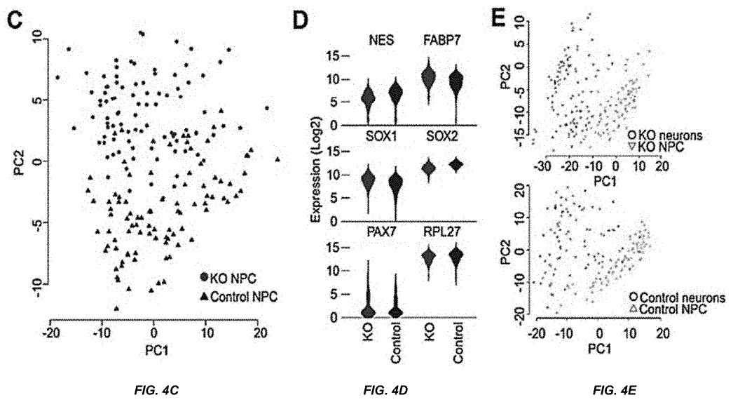

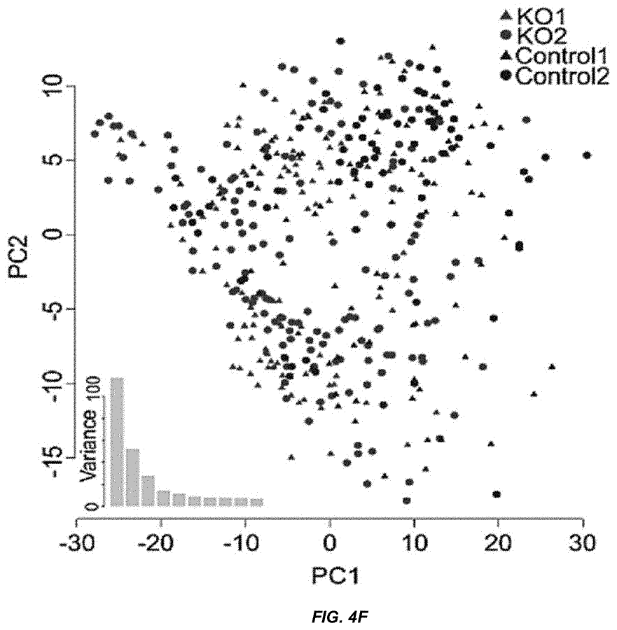

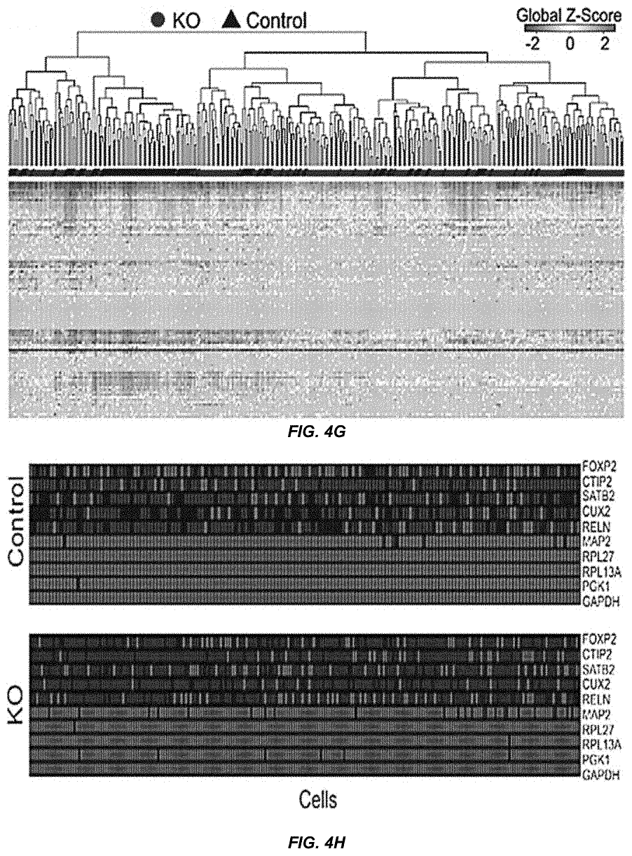

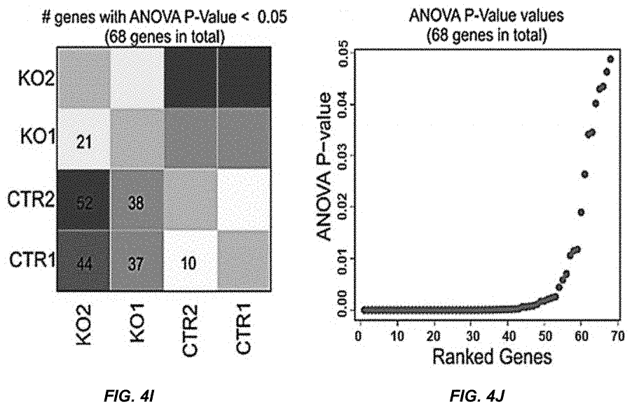

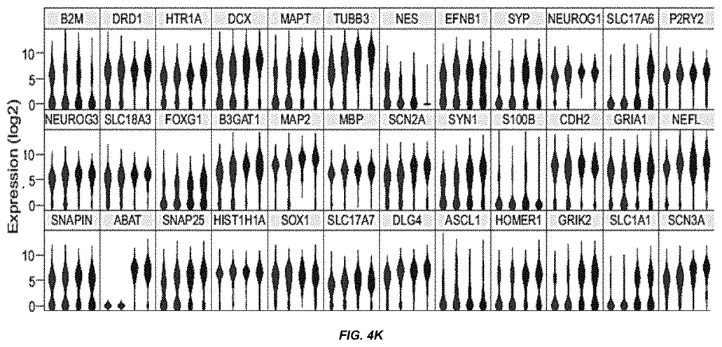

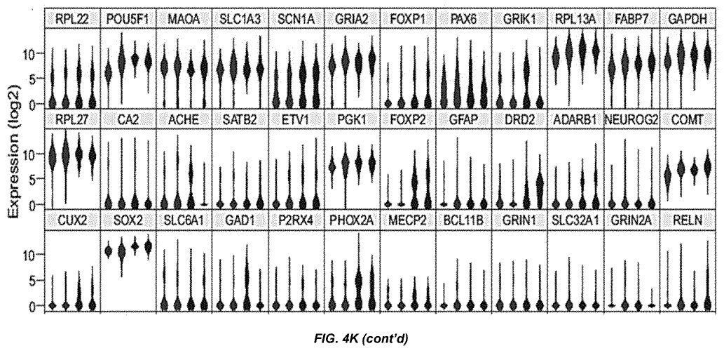

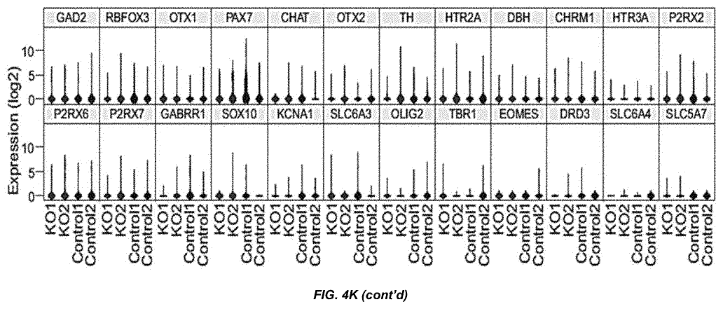

Since MeCP2 is an epigenetic regulator, changes in genetic expression resulting from its deficiency were investigated. PCR array (see FIG. 1C-D, and FIG. 4A-B) and single-cell qPCR (FIG. 1E-G, and FIG. 4C-F) analyses at timepoints throughout neuronal differentiation from neural progenitor cells (NPCs; day 0) revealed differences in genetic expression between MECP2-KO and control neurons. In agreement with the preeminent roles of MeCP2 in neuronal maturation and synaptic function, many of the implicated genes appeared to be relevant to synaptic function, and the difference in expression, for many genes, became increasingly pronounced as differentiation progressed (see FIG. 1C-G). The quantitation of cellular markers of neuronal fate and function in cells positive for the neuronal marker MAP2 revealed a decreased proportion of layer V and VI cortical neurons in MECP2-KO (P<0.0001) and alterations in neurotransmitter identity, such that the MECP2-KO populations of glutamatergic and cholinergic cells were decreased (P<0.0001; FIG. 1H, and FIG. 4G-K). Summary gene ontology analysis confirmed that many pathways affected by the KO are linked to synaptic structure or function (see FIG. 1I).

Artificial neural network modeling supports targeting synaptic dysfunction for treatment. Although synaptic pathology is a prominent consequence of MeCP2 deficiency, it is unclear that targeted treatment of synaptic dysfunction will yield measurably linked improvement in synaptic activity. Artificial neural networks offer a biologically plausible framework to explore how parameterized manipulation affects network activity. A neural network was generated in silico to predict the change in neuronal spiking activity expected to result from isolated rescue of synaptic morphology. Co-localized synaptic puncta values from Marchetto et al. were used as a proxy for synaptic knockdown. Holding all other parameters equivalent between KO and control, rescuing the synaptic defect in isolation sufficiently increased neuronal network activity (pairwise delta mean=0.877, range [−0.002, 7.981]; see FIG. 5A). The model thus supported using synaptic and neurotransmission phenotypes as actionable treatment targets to improve global network function.

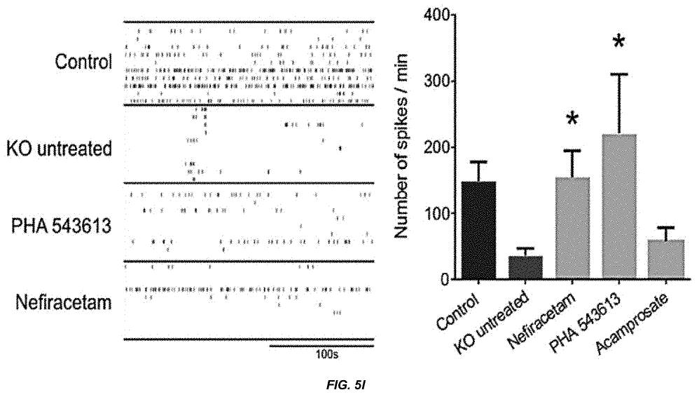

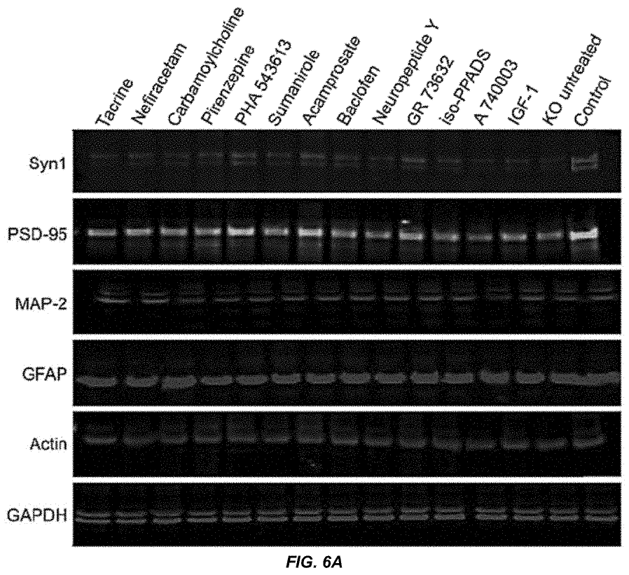

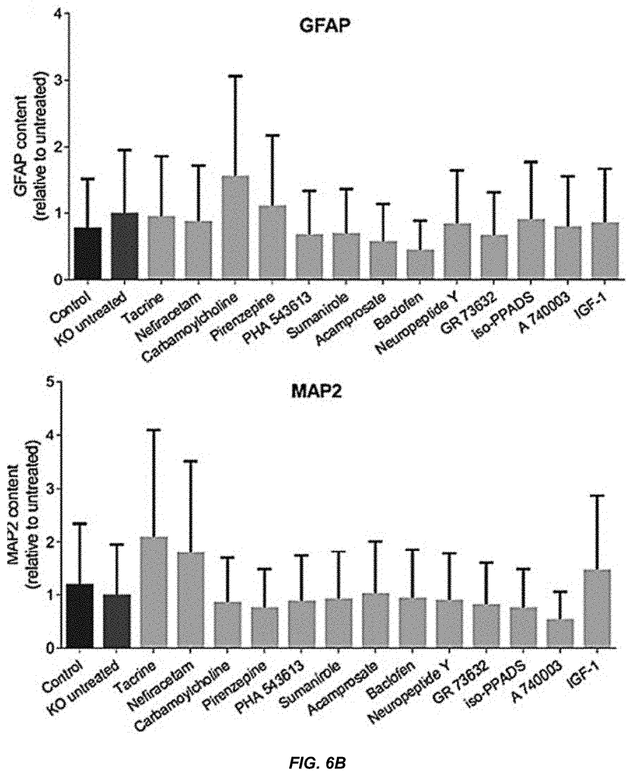

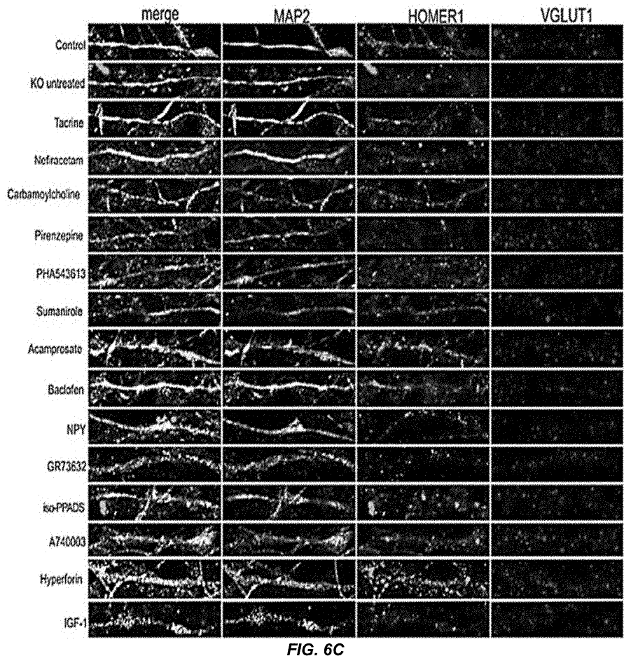

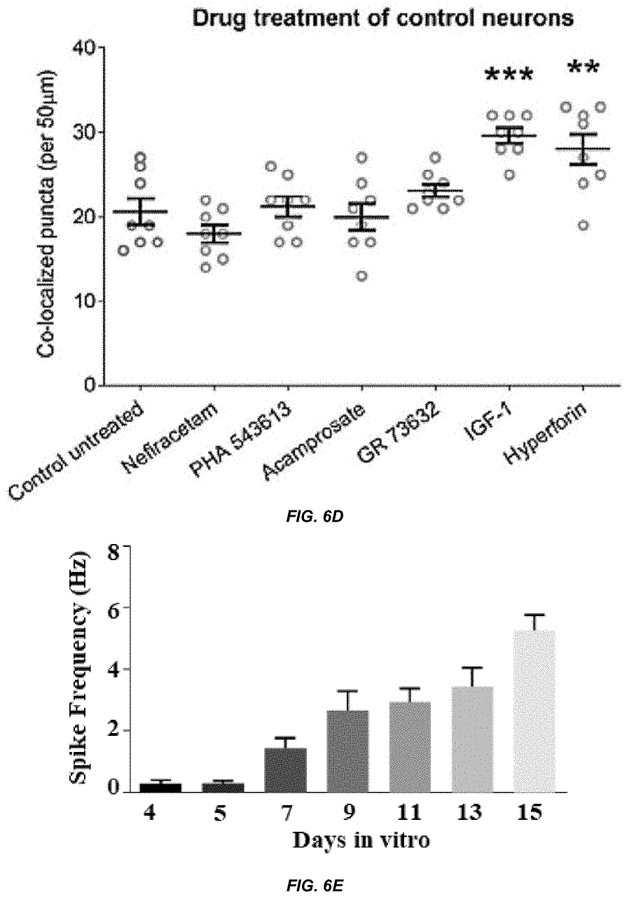

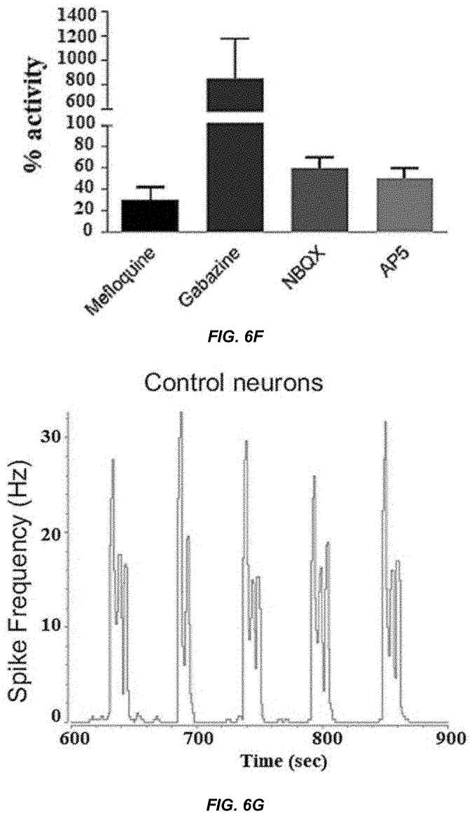

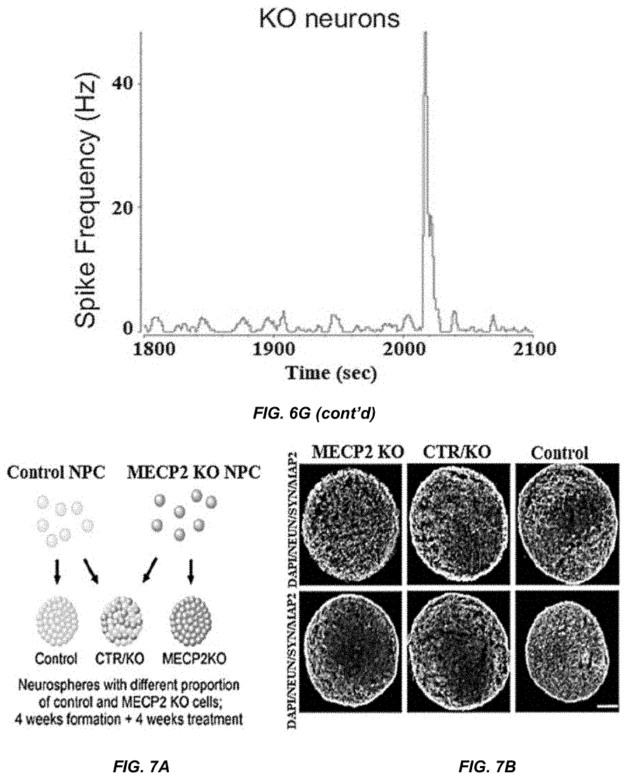

Pharmacologic screening identifies two compounds that specifically reverse MECP2-KO phenotypes. 14 commercially available pharmacologic compounds with mechanisms of action that counteract the synapse and neurotransmitter pathologies were selected and which are identified in FIG. 5B. For example, since MECP2-KO neurons exhibit cholinergic deficiency, compounds that promote this action were included (e.g., Tacrine, Carbamoylcholine). MECP2-KO and control neurons were treated with each of the 14 compounds and employed a tiered series of assays to identify those that could selectively rescue MECP2-KO pathology without affecting controls. Compounds that either did not treat pathology or significantly altered controls were eliminated. Nine of the 14 compounds increased the quantity of either the pre or postsynaptic protein Synapsin1 or PSD-95, respectively, in MECP2-KO neurons (see FIG. 5D-E, and FIG. 6A-B), the primary treatment endpoints for synaptogenesis. The capacity of these compounds to increase pre and post co-localized synaptic puncta in MECP2-KO neurons were evaluated, again eliminating the drugs that affected control neurons (see FIG. 5F-G, and FIG. 6C-D).

At the end of the selection process, three drugs were identified (Nefiracetam, Carbamoylcholine, and Acamprosate) that specifically increased synaptogenesis in human MECP2-KO neurons (see FIG. 2D-G). The large Carbamoylcholine metabolite, however, has limited gastrointestinal absorption and cannot traverse the blood-brain barrier. In consideration thereof, PHA 543613 was selected. PHA 543613, like Carboylcholine, is a direct cholinergic agonist that increases synaptic puncta with effect nearly identical to that of Carbamoylcholine (PHA 543613, 95% CI [−8.994, 0.4944] vs Carbamoylcholine, 95% CI [−9.619, −0.1306]) but has high oral bioavailability and rapid brain penetration in vivo.

Following successful synaptic improvement, the selected compounds (Nefiracetam, PHA 543613, and Acamprosate) were studied for their effects on synaptic function and network activity via calcium imaging and multi-electrode array (MEA) electrophysiology. Compared to controls, MECP2-KO neurons exhibit less frequent calcium oscillations (P<0.001), reduced calcium transients (P<0.001), and decreased MEA spike frequency (see FIG. 5H-I, and FIG. 6E-G). Only Nefiracetam and PHA 543613 could significantly increase the frequency of calcium oscillations, increase the percentage of neurons that are active (see FIG. 5H, and FIG. 6E-F), and restore the MEA spike frequency in MECP2-KO neurons (see FIG. 5I, and FIG. 6G). Since Nefiracetam and PHA 543613 selectively rescued synaptic morphology and activity in MECP2-KO neurons while leaving controls unaffected, they emerged as the most efficacious candidates for further testing.

Nefiracetam and PHA 543613 increase activity in MECP2-mosaic neurospheres. Because MECP2 resides on the X chromosome, random X-inactivation in females can produce a genetic pattern of mosaicism, with consequent phenotypic gradation, in which some cells express the normal allele while others are mutant. Therefore, as the next stage to evaluate the two drug candidates, a neurosphere model of MECP2 mosaicism was developed to mimic the female RTT brain by combining different proportions of control and MECP2-KO NPCs (see FIG. 7A-C). No difference was detected in size between untreated MECP2-mosaic (CTR/KO) neurospheres and controls (FIG. 7D), but both MECP2-KO (P<0.001) and MECP2-mosaic (P<0.001) neurospheres showed decreased calcium transient amplitude compared to controls (FIG. 7E). Treating MECP2-mosaic neurospheres with either Nefiracetam or PHA 543613 did not alter the diameter (P=0.79; FIG. 7F). Post-treatment cell viability (representing the inverse of drug-induced cytotoxicity) in the MECP2-mosaic neurospheres was improved in a dose-dependent manner by combined treatment with Nefiracetam and PHA 543613 (0.1 μM, 95% CI [−25.94, 0.10], P>0.05; 1 μM, 95% CI [−29.89, −3.85], P<0.01; 10 μM, 95% CI [−39.47, −13.44], P<0.001) (FIG. 7G). Calcium transient frequency in MECP2-mosaic neurospheres was increased by treatment with Nefiracetam in isolation (1 μM, P<0.05) and when combined with PHA 543613 (10 μM, P<0.001; FIG. 7H), but calcium transient amplitude was unaffected by either compound (P=0.255; FIG. 7I). To investigate the impact of the drug treatments on the electrophysiological properties of the MECP2-mosaic model, neurospheres treated with 1 μM Nefiracetam, 1 μM PHA 543613, or both Nefiracetam and PHA 543613 were plated to perform MEA analysis. Despite variability in the recordings, an inverse relationship of decreasing spike frequency with an increasing percentage of MECP2-KO cells in the neurospheres was observed, but this association did not reach significance (P=0.20). PHA 543613 and Nefiracetam each increased the overall spike count of MECP2-mosaic (CTR/KO) neurospheres after 5 weeks of treatment, but because MECP2-mosaic neurospheres exhibit similar spiking frequency as control neurospheres (P=0.65), these increases were likewise not significant (PHA 543613 vs CTR/KO, P=0.65; Nefiracetam vs CTR/KO, P=0.98; PHA+Nefi vs CTR/KO, P=0.86; FIG. 7J-K). Because each drug conferred a benefit to MECP2-mosaic neurospheres, both Nefiracetam and PHA 543613 were retained for further evaluation in a more complex human neurodevelopmental model.

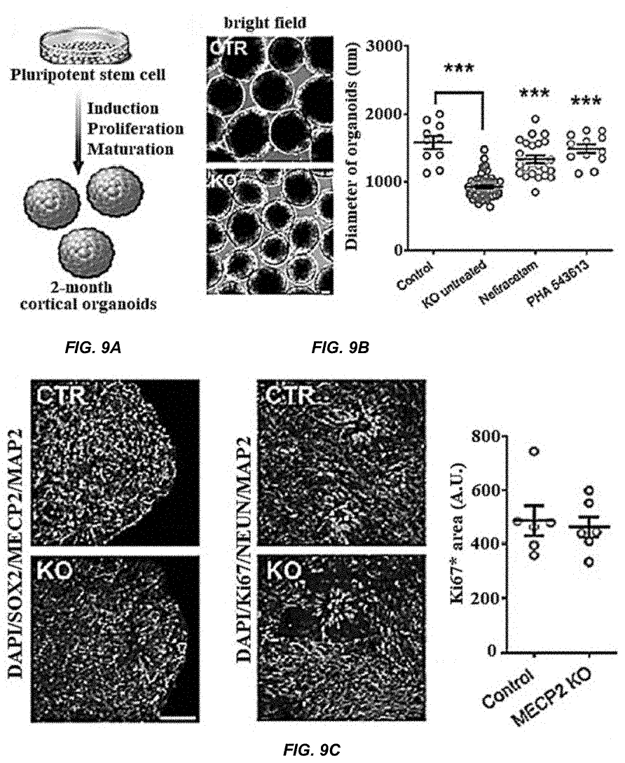

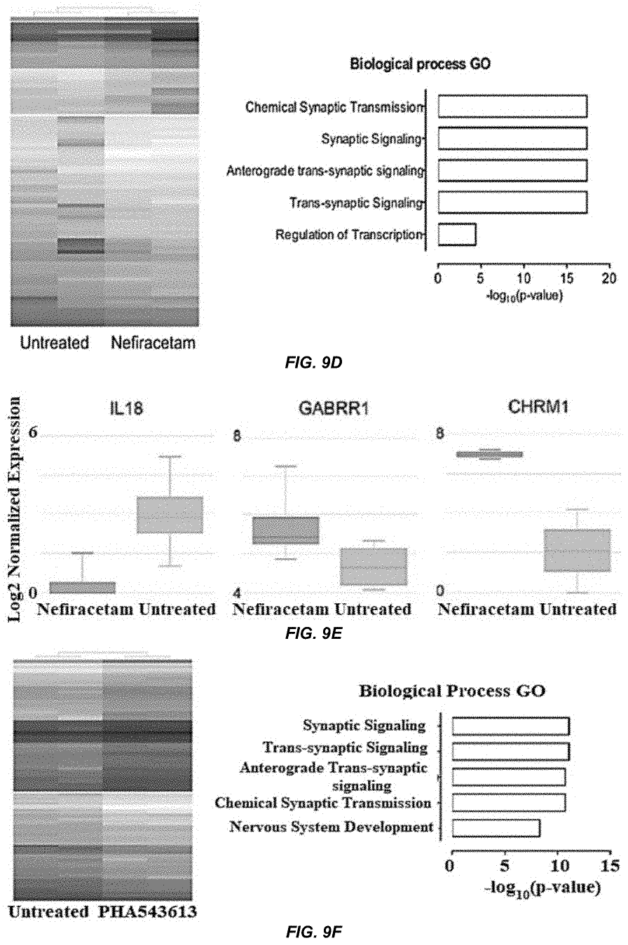

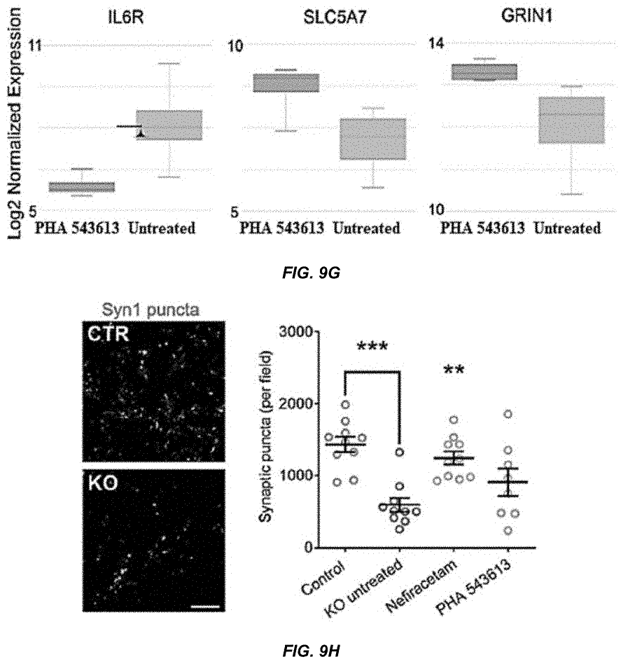

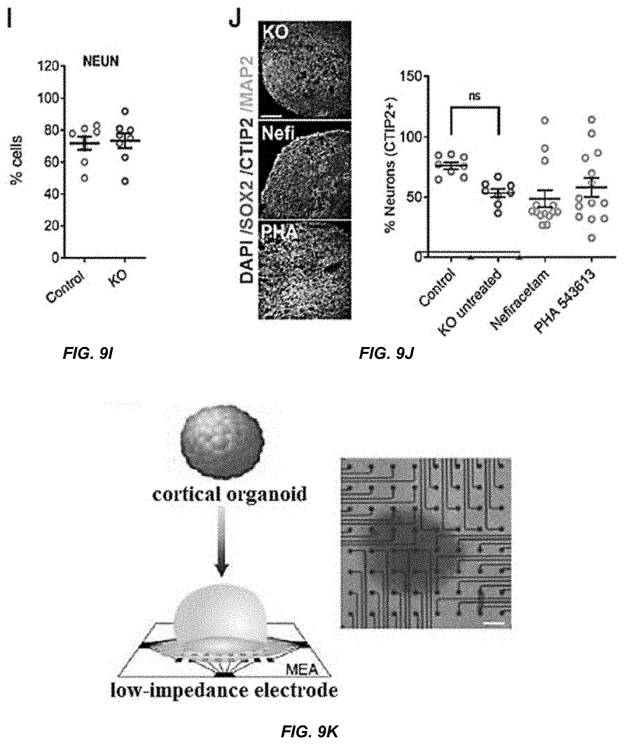

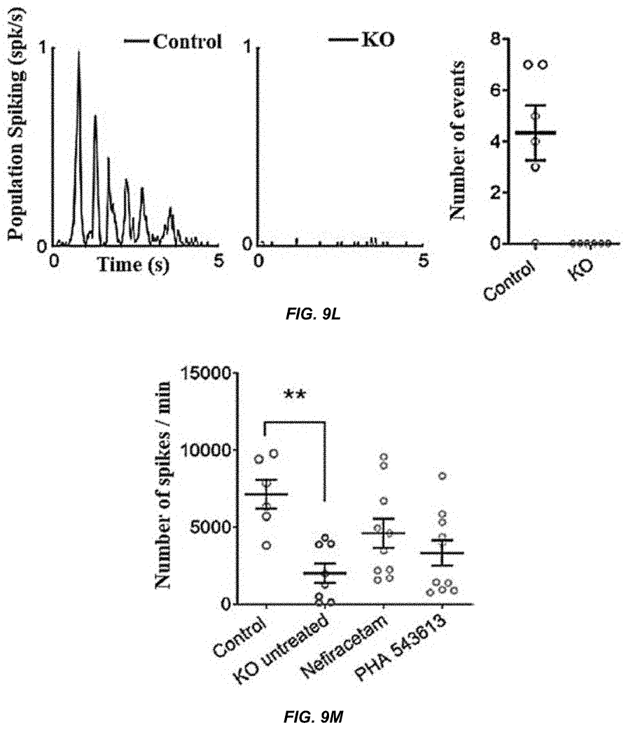

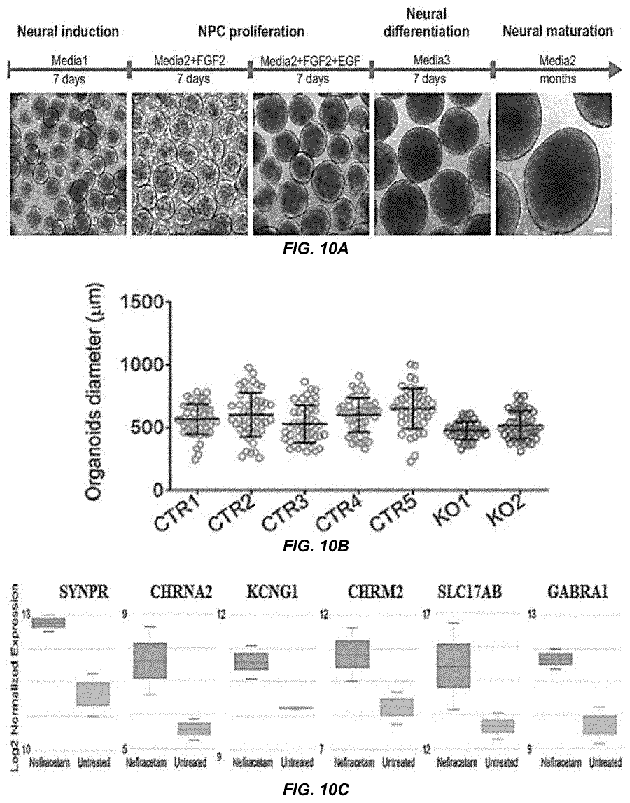

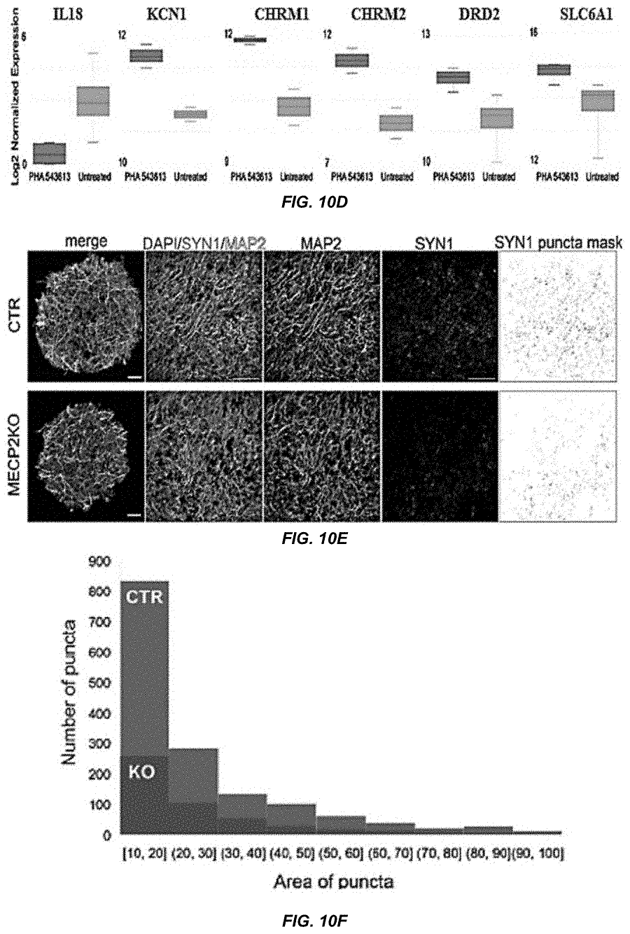

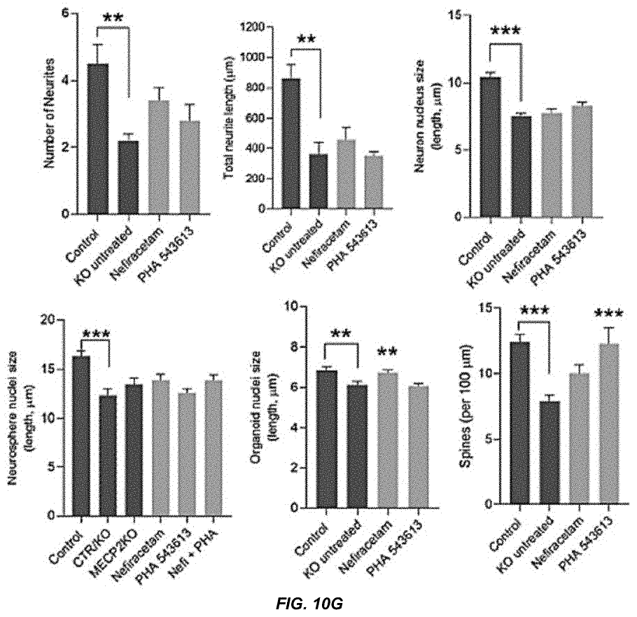

Nefiracetam and PHA 543613 rescue network activity in MECP2-KO cortical organoids. The therapeutic efficacy of Nefiracetam and PHA 543613 was evaluated with cortical organoids, a complex, three-dimensional human neurodevelopmental model that closely recapitulates aspects of human fetal neurodevelopment. MECP2-KO and control cortical organoids were generated, as described above (see FIG. 9A, and FIG. 10A-B). One-month old cortical organoids were treated for another month with either Nefiracetam (1 μM) or PHA 543613 (1 μM), and experiments and analyses were performed using organoids at 2-3 months of age. Both Nefiracetam (P<0.001) and PHA 543613 (P<0.001) increased the diameter of MECP2-KO organoids (FIG. 9B), which is reduced despite their equivalent areas of Ki67+ proliferation (P=0.73; FIG. 9C). RNA sequencing and gene ontology analyses in two-month old organoids revealed that both Nefiracetam and PHA 543613 promoted expression of genes involved in pathways relevant to synaptic function (FIG. 9D-G, FIG. 10C-D). The compounds were able to increase gene expression of specific neurotransmitter markers including cholinergic, GABAergic, and glutamatergic signaling. These findings were supported by the capacity of Nefiracetam (P<0.01), albeit not PHA 543613, to increase synaptic puncta (FIG. 9H, FIG. 10E-F). In 2-3-month-old MECP2-KO and control organoids, an equivalent percentage of cells immunostained positive for the neuronal marker NeuN (P=0.80; FIG. 9I), and the cortical layer V and VI marker CTIP2 was unaffected by drug treatment (P=0.07; FIG. 9J). Although the primary aim of the drug screening pipeline was to increase synaptogenesis and activity in MECP2-KO neurons, morphological features were also investigated. Nefiracetam and PHA 543613 did not increase the number or length of neurites and did not rescue nuclei size. However, an increase in neuronal spine-like protrusions was observed due to the treatments (FIG. 10G). To further evaluate network activity, cortical organoids were used to perform MEA electrophysiology. MECP2-KO organoids exhibited decreased population spiking compared to the controls (P<0.01), but treating the MECP2-KO organoids with Nefiracetam and PHA 543613 each increased population spiking to a level not significantly different from that of control organoids (Nefiracetam, P=0.98 in comparison to control organoids; PHA 543613, P=0.12 in comparison to control organoids; FIG. 9K-M).

It will be understood that various modifications may be made without departing from the spirit and scope of this disclosure. Accordingly, other embodiments are within the scope of the following claims.

1. A method for treating or rescuing synaptic defects caused by abnormal MeCP2 expression in a subject in need thereof, comprising administering to the subject a therapeutically effective amount(s) of a nootropic agent and/or an alpha-7 nicotinic acetylcholine receptor (α7-nAChR) agonist.

2. The method of claim 1, wherein the nootropic agent is a racetam-based nootropic agent.

3. The method of claim 2, wherein the racetam-based nootropic agent is selected from the group consisting of etiracetam, fasoracetam, levetiracetam, nebracetam, nefiracetam, piracetam, aniracetam, oxiracetam, pramiracetam and phenylpiracetam.

4. The method of claim 3, wherein the racetam-based nootropic agent is nefiracetam.

5. The method of claim 1, wherein the α7-nAChR agonist is selected from the group consisting of N-[(3R)-1-azabicyclo[2.2.2]oct-3-yl]furo[2,3-c]pyridine-5-carboxamide (PHA-543613), bradanicline, encenicline, N-[(3R,5R)-1-azabicyclo[3.2.1]oct-3-yl]furo[2,3-c]pyridine-5-carboxamide (PHA-709829), (4-bromophenyl) 1,4-diazabicyclo[3.2.2]nonane-4-carboxylate (SSR-180), tropisetron, N-[(3S)-1-azabicyclo[2.2.2]oct-3-yl]-1H-indazole-3-carboxamide hydrochloride (RG3487), 1-[6-(4-fluorophenyl)pyridin-3-yl]-3-(4-piperidin-1-ylbutyl) urea (SEN34625/WYE-103914), N-[4-(3-pyridinyl)phenyl]-4-morpholinepentanamide (SEN12333), 5-(6-[(3R)-1-azabicyclo[2,2,2]oct-3-yloxy]pyridazin-3-yl)-1H-indole (ABT-107), clozapine, 3-(2,4-dimethoxy-benzylidene)anabaseine (GTS-21), semapimod, and (2S)-2′H-spiro[4-azabicyclo[2.2.2]octane-2,5′-[1,3]oxazolidin]-2′-one (AR-R17779).

6. The method of claim 1, wherein the α7-nAChR agonist is PHA-543613.

7. The method of claim 1, wherein the nootropic agent and the alpha-7 nicotinic acetylcholine receptor (α7-nAChR) agonist are sequentially administered.

8. The method of claim 7, wherein a first pharmaceutical formulation comprises the nootropic agent and a second pharmaceutical formulation comprises the alpha-7 nicotinic acetylcholine receptor (α7-nAChR) agonist.

9. The method of claim 1, wherein the nootropic agent and the alpha-7 nicotinic acetylcholine receptor (α7-nAChR) agonist are concurrently administered.

10. The method of claim 9, wherein a single pharmaceutical composition formulated for oral delivery comprises the nootropic agent and the alpha-7 nicotinic acetylcholine receptor (α7-nAChR) agonist.

11. The method of claim 1, wherein the abnormal MeCP2 expression is cause by a genetic disease or disorder that affects the expression of the MECP2 gene.

12. The method of claim 11, wherein the genetic disease or disorder is selected from the group consisting of Rett syndrome, MECP2 duplication syndrome, neonatal encephalopathy, systemic lupus erythematosus (SLE), and autism spectrum disorder (ASD).

13. The method of claim 12, wherein the genetic disease or disorder is Rett syndrome.

14-15. (canceled)

16. The method of claim 1, wherein the subject is less than 25 years of age.

17. The method of claim 16, wherein the subject is less than 10 years of age.

18. A human-based neural drug screening platform for identifying therapeutic compounds that can ameliorate or rescue deleterious biological effect(s) resulting from abnormal expression of a gene from an X-chromosome, comprising:
(a) contacting set(s) of neurons, that have been differentiated from human stem cells, with a candidate drug, wherein a first set of neurons have been differentiated from human pluripotent stem cells that have mutation(s) affecting the normal expression of a gene from an X-chromosome and optionally, a second set of neurons that are differentiated from pluripotent stem cells that do not have said mutation(s);
(b) contacting set(s) of neurospheres, that have been generated from human stem cells, with the candidate drug, wherein a first set of neurospheres comprises neurospheres that have been differentiated from human pluripotent stem cells that have mutation(s) affecting the normal expression of the gene from an X-chromosome, and optionally, a second set of neurospheres that have been differentiated from the human pluripotent stem cells that do not have said mutation(s); and/or
(c) contacting set(s) of cortical organoids, that have been generated from human stem cells, with the candidate drug, wherein a first set of cortical organoids have been differentiated from human pluripotent stem cells that have mutation(s) affecting the normal expression of the gene from an X-chromosome, and optionally, a second set of cortical organoids that have been differentiated from the human pluripotent stem cells that do not have said mutation(s);
(d) evaluating whether the candidate drug rescues or ameliorates deleterious biological effect(s) resulting from abnormal expression of a gene from an X-chromosome in the first set of neurons, and/or the first set of neurospheres, and/or the first set of cortical organoids.

19. The human-based drug screening platform of claim 18, further comprising:
determining whether the candidate drug exhibits toxicity or negatively effects neural activity in the second set of neurons, and/or second set of neurospheres, and/or second set of cortical organoids.

20. The human-based drug screening platform of claim 18, wherein the mutation(s) affecting the normal expression of a gene from an X-chromosome are loss-of-function mutations.

21. The human-based drug screening platform of claim 18, wherein the gene is selected from MECP2, NLGN3, NLGN4, lysosomal alpha-galactosidase A, KDM6A, RPS6KA3, ABCD1, ATP7A, L1CAM, ATRX, and PHF8.

22. The human-based drug screening platform of claim 21, wherein the gene is MECP2.

23. The human-based drug screening platform of claim 18, wherein the biological activity of the neurons, neurospheres, and/or cortical organoids that is being determined comprises calcium oscillations, synaptic activity, and/or synaptic morphology.

24. The human-based drug screening platform of claim 18, wherein the first set of neurospheres is mosaic by further comprising neurospheres that have been differentiated from the human pluripotent stem cells that do not have said mutation(s).


SEQUENCE LISTING (3 sequences)

SEQ ID NO: 1 - DNA, 30 bp, Artificial Sequence
  Fragment of exon 3 MECP2 locus WT
tcccccaaacagcggcgctccatcatccgt

SEQ ID NO: 2 - DNA, 30 bp, Artificial Sequence
  Fragment of exon 3 MECP2 locus Q83X
tcccccaaatagcggcgctccatcatccgt

SEQ ID NO: 3 - DNA, 29 bp, Artificial Sequence
  Fragment of exon 3 MECP2 locus K82fs
tcccccaacagcggcgctccatcatccgt